(19) United States
(12) Patent Application Publication
(10) Pub. No.: US 2021/0041439 A1
(43) Pub. Date: Feb. 11, 2021

(54) SCREENING ASSAYS AND KITS FOR CHARACTERIZING AN ABILITY OF A CANDIDATE COMPOUND TO MODULATE A BINDING AFFINITY BETWEEN AN FBW7 PROTEIN AND AN FBW7 SUBSTRATE

(71) Applicants: FRED HUTCHINSON CANCER RESEARCH CENTER, Seattle, WA (US); UNIVERSITY OF WASHINGTON, Seattle, WA (US)

(72) Inventors: Bruce CLURMAN, Seattle, WA (US); Ning ZHENG, Seattle, WA (US); Shlomo HANDELI, Seattle, WA (US)

(21) Appl. No.: 17/046,984

(22) PCT Filed: Apr. 12, 2019
(86) PCT No.: PCT/US2019/027302

§ 371 Date: Oct. 12, 2020

Related U.S. Application Data
(60) Provisional application No. 62/747,036, filed on Oct. 17, 2018.
(60) Provisional application No. 62/657,581, filed on Apr. 13, 2018.

(51) Int. Cl. G01N 33/573; G01N 33/543; G01N 21/76

(52) U.S. Cl. CPC G01N 33/573; G01N 33/54393; G01N 2500/02; G01N 2333/9015; G01N 21/76

(57) ABSTRACT

Screening assays and related reagent kits for characterizing an ability of a candidate compound to modulate a binding affinity between a mutant F-box/WD repeat-containing protein 7 (FBW7) peptide and an FBW7 substrate are described.

### CROSS-REFERENCES TO RELATED APPLICATIONS

This application claims the benefit of U.S. Provisional Application No. 62/657,581, filed Apr. 13, 2018, and U.S. Provisional Application No. 62/747,036, filed Oct. 17, 2018, the contents of which are hereby incorporated by reference in their entireties.

### STATEMENT REGARDING SEQUENCE LISTING

The sequence listing associated with this application is provided in text format in lieu of a paper copy and is hereby incorporated by reference into the specification. The name of the text file containing the sequence listing is 68324_Seq_Listing_Final_2019-04-11.txt. The text file is 122 KB; was created on Apr. 11, 2019; and is being submitted via EFS-Web with the filing of the specification.

### STATEMENT OF GOVERNMENT LICENSE RIGHTS

This invention was made with government support under CA178143 awarded by the National Institutes of Health. The government has certain rights in the invention.

### BACKGROUND

The ubiquitin-proteasome system (UPS) targets proteins for degradation and controls many biological processes. UPS specificity is conferred by E3 ubiquitin ligases, such as F-box/WD repeat-containing protein 7 (FBW7, see UniProt number Q969H0, OMIM No. 606278, SEQ. ID NO. 1), which recognize protein targeted for degradation, often in response to substrate modification. Mis-regulated UPS activity contributes to many diseases, including cancer. Tumors contain mutations that either enhance or disable UPS function, depending on the affected gene. The UPS is thus an important therapeutic target in cancer and UPS inhibitors have already impacted clinical care. Current therapeutic approaches involve inhibitors to UPS components; however, these approaches cannot be applied to UPS components that contain amino acid substitutions at key binding sites that prevent binding of the UPS inhibitor. Indeed, several key tumor suppressors are E3 ligases with mutations. These mutated E3 ligases have reduced activity compared to wild-type due to missense mutations that reduce their affinity for substrates, thus allowing oncogenic substrates to accumulate.

The FBW7 protein is an E3 ligase that targets critical proteins (e.g. c-Myc, Notch, cyclin E, and Jun) for degradation, many of which have been implicated as oncoproteins in certain disease settings. Mutated Fbw7 is a commonly mutated tumor suppressors and its prevalence is in approximately 10% of all cancers and has higher prevalence in T-cell acute lymphoblastic lymphomas. See, for example, Davis et al., Cancer Cell, 2014 Oct. 13; 26(4): 455-464, the content of which is incorporated herein by reference in its entirety. Because of FBW7's critical role in the regulation of degradation and cell proliferation and because its mutation frequently leads to cancer and other FBW7-mediated malignancies, there is a need for treatments of such cancers and FBW7-mediated malignancies. The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In an aspect, the present disclosure provides methods of characterizing an ability of a candidate compound to modulate a binding affinity between a mutant FBW7 protein and an FBW7 substrate.

In another aspect, the present disclosure provides kits suitable for characterizing an ability of a candidate compound to modulate a binding affinity between a mutant FBW7 protein and an FBW7 substrate.

### DESCRIPTION OF THE DRAWINGS

The foregoing aspects and many of the attendant advantages of the claimed subject matter will become more readily appreciated as the same become better understood by reference to the following detailed description, when taken in conjunction with the accompanying drawings, wherein:

The foregoing aspects and many of the attendant advantages of claimed subject matter will become more readily appreciated as the same become better understood by reference to the following detailed description, when taken in conjunction with the accompanying drawings, wherein:

FIG. 1A provides chemical structures of compounds, in accordance with an embodiment of the disclosure;

FIG. 1B graphically illustrates dose response of the compounds of FIG. 1A in an ALPHAscreen using cyclin E peptide substrate, in accordance with an embodiment of the disclosure;

FIG. 1C shows in vitro ubiquitylation assay results using recombinant wild-type F-box/WD repeat-containing protein 7 (FBW7) and mutated FBW7R505L (FBW7^{ARG}) dimers in the presence of DMSO only and compounds of FIG. 1A, in accordance with an embodiment of the disclosure. Data shows reconstitution of full-length substrate ubiquitylation of FBW7^{ARG} on FBW7 target substrates Cyclin E, Jun, and Notch in the presence of the compounds of FIG. 1A;

FIG. 2 schematically illustrates (left) a mutant FBW7 protein (“FBW7^{ARG}”) having a reduced binding affinity for a substrate in the absence of an agonist according to an embodiment of the present disclosure and (right) the substrate binding to the FBW7^{ARG} with the administration of the agonist wherein the substrate is ubiquitylated, in accordance with an embodiment of the disclosure;

FIG. 3 schematically illustrates (left) binding of a substrate to wildtype E3 ubiquitin ligase, (middle) failure of a substrate to bind to a mutated E3 ubiquitin ligase; and (right) binding of a substrate to a mutated E3 ubiquitin ligase in the presence of an agonist according to an embodiment of the present disclosure;

FIG. 4A schematically illustrates a screening assay, in accordance with an embodiment of the disclosure;

FIG. 4B schematically illustrates another screening assay, in accordance with embodiments of the disclosure. A his-tagged mutant FBW7 (FBW7^{ARG}) is bound to a nickel acceptor bead, whereas a biotinylated peptide is bound to a streptavidin donor bead. A candidate compound that increases FBW7^{ARG}-cyclin E binding is depicted coupling the Nickel acceptor bead to the streptavidin donor bead;

FIG. 5A graphically illustrates a GST-FBW7 construct including a TEV site and a linker, in accordance with an embodiment of the disclosure;

FIG. 5B graphically illustrates schematic of cleavage of GST from FBW7-Skp 1 by TEV protease;

FIG. 5C shows efficient cleavage of GST-FBW7 by TEV protease;

FIG. 5D shows purification of homogenous dimeric Skp1-FBW7 by anion exchange and size exclusion chromatography. Left: Representative trace of FBW7 dimer on anion exchange using a 1 ml SourceQ column. The gradient elution carried the concentrations of two Tris/DTT buffers of different ionic strengths (0 M and 1 M NaCl). Right: Representative trace of FBW7 dimer on 25 ml Superdex 200 column. The Skp1-FBW7 peak corresponds to 170 kDa, consistent with a dimeric complex;

FIG. 5E shows in vitro ubiquitylation of cyclin E with recombinant FBW7 or FBW7^{ARG}, in accordance with an embodiment of the disclosure. Reactions contained E1, Nedd8-Cull, hCdc34, Ubiquitin, ATP, and buffer (min=minutes);

FIG. 6A graphically illustrates the impact of cyclin E peptide concentration on screening assay signal obtained with wt-FBW7 and FBW7^{ARG}, in accordance with an embodiment of the disclosure;

FIG. 6B graphically illustrates the impact of pH and reducing agent on screening assay signal (wt-FBW7), in accordance with an embodiment of the disclosure;

FIG. 6C graphically illustrates that Wt-FBW7 produces screening assay signal is approximately 100-fold greater with phosphorylated cyclin E peptide compared with unphosphorylated peptide, whereas FBW7^{ARG} (RL-FBW7) produces an intermediate signal with phosphorylated peptide;

FIG. 7A graphically illustrates data from assay plates (n=12, triplicate results), including controls. Large separation in activity (Z′=0.95+0.01, S:B=14.6±2.3) between wells dosed with wild type FBW7 (high control) and mutant FBW7 (low control) indicate an outstanding HTS assay. Assay results for methylene blue, dosed at 10 μM, also plotted;

FIG. 7B is a representative graph of compound activity correlating plate replicas. The best fit line has an r=0.998, indicative of the assay's reproducibility;

FIG. 8 graphically illustrates a 10K miniaturized FBW7 agonist pilot;

FIG. 9 graphically illustrates an overview of primary screen and counter-screening results, in accordance with an embodiment of the disclosure. The initial screening assay was performed with a compound library in 1536-well format using purified FBW7^{ARG} and cyclin E peptide. Counterscreen 1 utilized only FBW7 protein and Counterscreen 2 utilized only cyclin E peptide. 41 compounds are potential selective agonists of FBW7^{ARG};

FIG. 10A graphically illustrates distribution of hits of a screening assay, in accordance with an embodiment of the disclosure, with >10% activity;

FIG. 10B graphically illustrates FBW7 agonist dose responses for 3 compounds, in accordance with embodiments of the disclosure;

FIG. 11 graphically illustrates hit validation where the Y-axis plot is expanded to highlight the baseline relative to WT-FBW7. The dashed horizontal line indicates 3 standard deviations plus an average of the DMSO control. Results greater than the dashed line are considered active;

FIG. 12A provides chemical structures of two compounds, in accordance with embodiments of the disclosure, with corresponding screening assay activity shown;

FIG. 12B shows cyclin E is stabilized in Hct116 cells with a homozygous FBW7R505C to the same extent as in FBW7 null Hct116 cells; and

FIG. 13 is a schematic block diagram of a method for characterizing an ability of a candidate compound to modulate a binding affinity between a mutant FBW7 protein and an FBW7 substrate, in accordance with an embodiment of the disclosure.

### DETAILED DESCRIPTION

The present disclosure provides E3 ubiquitin ligase agonists, pharmaceutical compositions including the E3 ubiquitin ligase agonists, related methods of use, screening assays for identifying E3 ubiquitin ligase agonists, and kits suitable for performing screening assays for identifying E3 ubiquitin ligase agonists. In a particular embodiment, the present disclosure provides small molecule F-box/WD repeat-containing protein 7 (FBW7) agonists that restore interactions between mutant FBW7 proteins and substrates thereof. As discussed further herein, in an embodiment, the small molecule E3 ubiquitin ligase agonists and pharmaceutical compositions of the present disclosure functionally restore function of mutant FBW7 proteins found in cancers. Importantly, such small molecule E3 ubiquitin ligase agonists and pharmaceutical compositions allow mutant FBW7 proteins to ubiquitylate their target substrates to nearly the same or the same extent as wild-type FBW7 proteins. Accordingly, in certain aspects, the present disclosure also provides methods of treating FBW7-mediated malignancies with the compounds and/or pharmaceutical compositions of the present disclosure. As discussed further herein, the present disclosure further provides screening assays for selecting and/or identifying E3 ubiquitin ligase agonists.

### Screening Assays

In an aspect, the present disclosure provides an assay for characterizing an ability of a candidate compound to modulate a binding affinity between a mutant FBW7 protein and an FBW7 substrate. Candidate compounds that increase the binding affinity between a mutant FBW7 protein and an FBW7 substrate may be useful in treating diseases including FBW7-mediated malignancies, such as FBW7-mediated cancers.

In an embodiment, the method generally includes incubating the candidate compound with a donor configured to generate a reactive oxygen species when in an excited state and with an acceptor configured to generate luminescent light when the acceptor is in proximity to the reactive oxygen species; wherein either the donor or the acceptor is associated with the mutant FBW7 protein and the other of the acceptor or the donor is associated with the FBW7 substrate; exciting the donor to generate the reactive oxygen species; and measuring an amount of luminescent light generated by the acceptor in response to the reactive oxygen species. As used herein, “FBW7 protein” refers to portions of FBW7 proteins including peptide fragments and derivatives thereof, as well as full-length FBW7 proteins.

As used herein, a “mutant FBW7 protein” refers to an oligopeptide, polypeptide, or protein including one or more mutations from wildtype FBW7 proteins. In an embodiment, the mutant FBW7 protein includes an FBW7 substrate-binding interface and or one or more mutations from a wildtype sequence. In an embodiment, the mutant FBW7 protein includes one or more missense mutations in an FBW7 substrate-binding interface that reduce the binding affinity of mutant FBW7 protein for the FBW7 substrate relative to a binding affinity of a wildtype FBW7 protein for the FBW7 substrate. In an embodiment, the mutant FBW7 protein includes a mutation selected from the group consisting of R465, R479, R505, R689 relative to a wildtype FBW7 protein, such as SEQ ID NO. 1, and combinations thereof.

In an embodiment, the method includes incubating the candidate compound with a donor bead configured to generate a reactive oxygen species when in an excited state and with an acceptor bead configured to generate luminescent light when the acceptor bead is in proximity to the reactive oxygen species; wherein either the donor bead or the acceptor bead is coupled to the mutant FBW7 protein and the other of the acceptor bead or the donor bead is coupled to the FBW7 substrate; exciting the donor bead to generate the reactive oxygen species; and measuring an amount of luminescent light generated by the acceptor bead in response to the reactive oxygen species. In an embodiment, if the amount of luminescent light is different from an amount of control luminescent light, a modulated binding affinity between the mutant FBW7 protein and the FBW7 substrate is indicated.

As above, the donor bead is configured to generate a reactive oxygen species when in an excited state. In an embodiment, the donor bead comprises sensitizer configured to generate the reactive oxygen species when the sensitizer is in an excited state. In an embodiment, the sensitizer is a photosensitizer configured to generate the reactive oxygen species when the sensitizer is illuminated with stimulation electromagnetic radiation. Such stimulation electromagnetic radiation can include, among others, ultraviolet light, visible light, infrared light, and combinations thereof. In an embodiment, the photosensitizer is phthalocyanine. In an embodiment, such illumination magnetic light transitions the photosensitizer to the excited state, such as an electronic state energetically above an electronic ground state.

As user herein, a “reactive oxygen species” refers to chemically reactive chemical species containing oxygen. In an embodiment, the reactive oxygen species is selected from the group consisting of peroxides, superoxide, hydroxyl radicals, singlet oxygen, alpha-oxygen, and combinations thereof. In an embodiment, the reactive oxygen species is singlet oxygen. As discussed further herein, in certain embodiments, the acceptor is configured to generate a signal in response to the reactive species, such as singlet oxygen.

The acceptor, such as an acceptor bead, is configured to generate luminescent light when the acceptor bead is in proximity to the reactive oxygen species. As discussed further herein, such proximity to the reactive oxygen species may be due to, for example, binding or coupling of the donor and acceptor beads mediated by the candidate compound.

In an embodiment, the acceptor is configured to react with the reactive oxygen species to generate the luminescent light. Accordingly, in an embodiment, the acceptor is configured to generate the luminescent light when the acceptor is close enough to a donor in an excited state to contact the reactive oxygen species before the reactive oxygen species reacts with other species. In an embodiment, a distance between the donor and the acceptor is less than or equal to a diffusion length of a reactive oxygen species half-life. As discussed below, singlet oxygen diffuses approximately 200 nm within its 4-microsecond half-life. In that regard, acceptors within about 200 nm of an excited donor are positioned to contact the singlet oxygen reactive species from a donor coupled thereto through an agonist and generate luminescent light in response.

In an embodiment, the acceptor comprises a luminescent compound configured to generate luminescent light when the luminescent compound is in proximity to the reactive oxygen species. Without wishing to be bound by theory, it is believed that the luminescent compound interacts with the reactive oxygen species to generate luminescent light. In an embodiment, the luminescent compound is selected from thioxene, anthracene, rubrenein, and combinations thereof.

The methods of the present disclosure include mutant FBW7 protein coupled to either the donor or the acceptor. As discussed further herein, such mutant FBW7 proteins generally have a reduced binding affinity for FBW7 substrates. Certain candidate compounds will increase the binding affinity of mutant FBW7 proteins for their substrates, thus, on average, binding more mutant FBW7 proteins to their respective substrates after incubation of the mutant FBW7 protein with the candidate compound.

In an embodiment, the mutant FBW7 protein can be coupled to either a donor bead or an acceptor bead. In an embodiment, such coupling includes covalent bonds. In an embodiment, such coupling includes non-covalent bounds. In an embodiment, the mutant FBW7 protein includes a terminal polyhistadine tag, such as a terminal hexahistadine sequence, and either the donor bead or the acceptor bead includes one or more metal ions configured to coordinate with the polyhistadine tag to couple the mutant FBW7 protein to the donor or the acceptor bead. In an embodiment, the one or more metal ions are selected from the group consisting of copper ions, nickel ions, zinc ions, cobalt ions, and combinations thereof. In an embodiment, the mutant FBW7 includes one or more biotin moieties and the donor or the acceptor bead includes one or more avidin moieties, such as one or more streptavidin moieties. In an embodiment, the mutant FBW7 protein includes one or more avidin moieties, such as one or more streptavidin moieties, and either the donor or the acceptor bead includes one or more biotin moieties. In this regard, the mutant FBW7 protein is configured to couple with either the donor bead or the acceptor bead.

As used herein, “an FBW7 substrate” refers to a molecule, such as a protein, on which an FBW7 protein naturally acts, such as to ubiquitylate the molecule. In an embodiment, the FBW7 substrate is a cognate substrate. The FBW7 substrates are in contrast to neo-substrates of FBW7 proteins on which an FBW7 protein does not naturally act.

As discussed further herein, the FBW7 substrate is coupled to whichever of the donor or the acceptor is not coupled to the mutant FBW7 protein. In this regard, the donor and the acceptor are brought into proximity as the mutant FBW7 protein and the FBW7 substrate couple, such as when the coupling is mediated by a candidate compound. In an embodiment, the FBW7 substrate and the mutant FBW7 protein are not coupled to the same bead, whether it is the donor bead or the acceptor bead. In an embodiment, the FBW7 substrate is coupled to a surface of the donor bead; and wherein the mutant FBW7 protein is coupled to a surface of the acceptor bead. In another embodiment, the FBW7 substrate is coupled to a surface of the acceptor bead; and wherein the mutant FBW7 protein is coupled to a surface of the donor bead.

The FBW7 substrate can be any substrate upon which an FBW7 protein acts, including the mutant FBW7 protein. In an embodiment, the FBW7 substrate is selected from the group consisting of c-Myc, n-Myc, Notch, cyclin E, c-Jun, PGC-1a, SREBP1, SREBP2, MCL1, MED13/13L, KLF5, KLF2, C/EBδ, TGIF1, GATA2, GATA3, KLG10, KLF11, and C/EBPalpha. In an embodiment, the FBW7 substrate is selected from the group consisting of proteins according to one or more of SEQ ID NOS. 2-20. In an embodiment, the FBW7 substrate is cyclin E, such as according to SEQ ID NO. 5.

As above, the FBW7 substrate can be coupled to either the donor or the acceptor. In an embodiment, such coupling includes covalent bonds. In an embodiment, such coupling includes non-covalent bounds. In an embodiment, the FBW7 substrate includes a terminal polyhistadine tag, such as a terminal hexahistadine sequence, and either the donor bead or the acceptor bead includes one or more metal ions configured to coordinate with the polyhistadine tag to couple the FBW7 substrate to the donor bead or the acceptor bead. In an embodiment, the one or more metal ions are selected from the group consisting of copper ions, nickel ions, zinc ions, cobalt ions, and combinations thereof. In an embodiment, the metal is nickel. In an embodiment, the FBW7 substrate includes one or more biotin moieties and the donor or the acceptor bead includes one or more avidin moieties, such as one or more streptavidin moieties. In an embodiment, the FBW7 substrate includes one or more avidin moieties, such as one or more streptavidin moieties, and either the donor or the acceptor bead includes one or more biotin moieties. In this regard, the FBW7 substrate is configured to couple with either the donor bead or the acceptor bead.

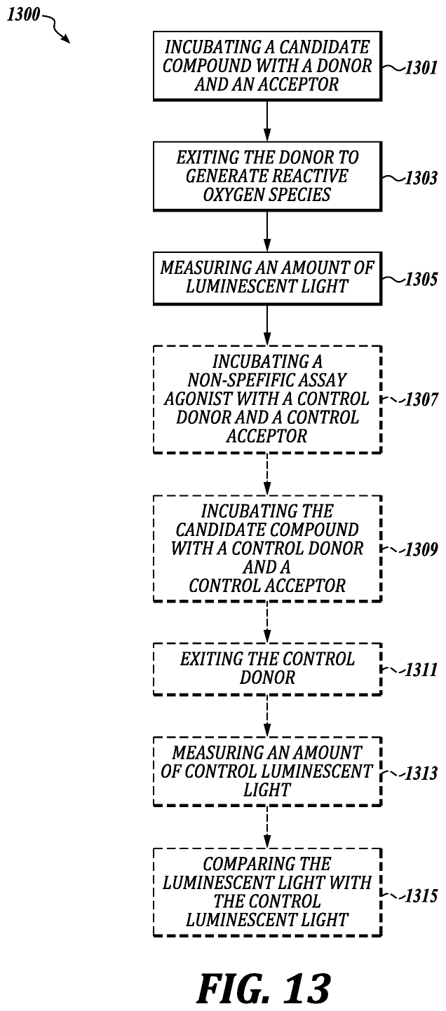

An embodiment of a method 1300, in accordance with an embodiment of the disclosure, will now be described. Reference is made to FIG. 13, in which method 1300 is schematically illustrated. In an embodiment, the method 1300 begins with process block 1301, which includes incubating a candidate compound with a donor and an acceptor. As above, in an embodiment, incubating the candidate compound with a donor and an acceptor includes incubating the candidate compound with a donor configured to generate a reactive oxygen species when in an excited state and with an acceptor configured to generate luminescent light when the acceptor is in proximity to the reactive oxygen species; wherein either the donor or the acceptor is coupled to the mutant FBW7 protein and the other of the acceptor or the donor is coupled to the FBW7 substrate.

In an embodiment, incubation includes contacting the candidate compound with the donor and the acceptor. The candidate compound may be contacted with the donor bead and the acceptor bead in any order. In an embodiment, the candidate compound is contacted with the donor bead first and then contacted with the acceptor bead. In an embodiment, the candidate compound is contacted by the acceptor bead first and then contacted with the donor bead. In an embodiment, the candidate compound is contacted with the donor bead and the acceptor bead simultaneously.

In an embodiment, incubation includes: contacting the candidate compound with the acceptor under conditions and for a time sufficient to allow for coupling of the candidate compound and the acceptor; and contacting the candidate compound with the donor under conditions and for a time sufficient to allow for coupling of the candidate compound and the donor. In an embodiment, contacting the candidate compound with the acceptor bead includes contacting the candidate compound with the acceptor bead for an acceptor-bead incubation time in a range of about 10 minutes to about one hour. In an embodiment, contacting the candidate compound with the donor bead includes contacting the candidate compound with the donor bead for a donor-bead incubation time in a range of about 4 hours to about 20 hours.

In an embodiment, contacting the candidate compound with the donor bead and the acceptor bead includes contacting the candidate compound in a medium, such as a solution or suspension having a pH in a range of about 6.0 to about 8.0. In an embodiment, contacting the candidate compound with the donor bead and the acceptor bead includes contacting the candidate compound in a medium, such as a solution or suspension having a pH in a range of about 6.5 to about 7.5. As discussed further herein, such pH ranges generally provide assay conditions generally resulting relatively high amounts of luminescent light in response to successful candidate compound-mediated coupling between the donor bead and the acceptor bead.

In an embodiment, process block 1301 is followed by process block 1303, which includes exciting the donor to generate a reactive oxygen species. As above, in certain embodiments, the donor includes a photosensitizer. Accordingly, in certain embodiments, exciting the donor bead includes illuminating the photosensitizer with stimulation electromagnetic radiation to place the photosensitizer in an excited state and generate the reactive oxygen species. While excitation with illumination light is described elsewhere herein, it will be understood that other forms of excitation are possible, such as electrical excitation, electrochemical excitation, and the like.

Process block 1303 can be followed by process block 1305, which includes measuring an amount of luminescent light generated by the acceptor bead in response to the reactive oxygen species. Measuring the luminescent light can be performed according to methods known in the art, such as with a photodetector.

In an embodiment, the amount of luminescent light generated by the acceptor bead is measured relative to an amount of luminescent light generated by a control assay.

Accordingly, in an embodiment, process block 1305 can be followed by process block 1307, which includes incubating a non-specific assay agonist with a control donor and a control acceptor. In an embodiment, process block 1307 includes incubating a non-specific assay agonist with a control donor bead configured to generate a control reactive oxygen species when in an exited state and with a control acceptor bead configured to generate control luminescent light when the control acceptor bead is in proximity to the control reactive oxygen species; wherein either the control donor bead or the control acceptor bead is coupled to a mutant FBW7 protein and the other of the control acceptor bead or the control donor bead is coupled to an FBW7 substrate. In an embodiment, the mutant FBW7 protein, the FBW7 substrate, the donor bead, and/or the acceptor bead are the same in the control assay as is other portions of the method, such as in process blocks 1301-1305. In an embodiment, the non-specific assay agonist is methylene blue. As discussed further herein, the control assay including a non-specific assay agonist provides a baseline of luminescent light from which an experimental amount of luminescent light may be measured. In an embodiment, process block 1307 is optional.

In an embodiment, process block 1305 or process block 1307 is followed by process block 1309, which includes incubating the candidate compound with a wildtype FBW7 protein, such as a peptide according to SEQ ID NO. 1. In that regard, a comparison may be made between assays in which the candidate compound is incubated with the mutant FBW7 protein and the wildtype FBW7 protein. As discussed further herein, mutant FBW7 proteins generally have reduced binding affinity for their substrate in the absence of an agonist. Accordingly, in an embodiment, process block 1309 generally includes incubating the candidate compound with a control donor bead configured to generate a control reactive oxygen species when in an exited state and with a control acceptor bead configured to generate control luminescent light when the control acceptor bead is in proximity to the control reactive oxygen species; wherein either the control donor bead or the control acceptor bead is coupled to a wild type FBW7 protein and the other of the control acceptor bead or the control donor bead is coupled to an FBW7 substrate. In an embodiment, the FBW7 substrate is unphosphorylated cyclin E. In an embodiment, process block 1309 is optional.

In an embodiment, process block 1307 or process block 1309 is follow by process block 1311, which includes exciting the control donor to generate the control reactive oxygen species. In an embodiment, exciting the control donor is performed under the same or similar conditions as exciting the donor, such as in process block 1303. In that regard, analogous conditions are used in both scenarios making a direct comparison between the assay and the control assay easier. In an embodiment, process block 1311 is optional.

In an embodiment, process block 1311 is follow by process block 1313, which includes measuring the amount of control luminescent light, such as measuring the amount of control luminescent light generated by the control acceptor in response to the control reactive oxygen species. Measuring the amount of control luminescent light can be according to the methods and with equipment used to measuring the amount of luminescent light, such as according to process block 1305. In an embodiment process block 1313 is optional.

In an embodiment, process block 1313 is followed by process block 1315, which includes comparing the amount of luminescent light, such as luminescent light measured in process block 1305, with the amount of control luminescent light, such as control luminescent light measured in process block 1313. In an embodiment, process block 1315 includes comparing the amount of luminescent light generated by the acceptor in response to the reactive oxygen species and in the presence of the candidate compound and the amount of control luminescent light generated by the control acceptor bead in response to the control reactive oxygen species. Such a comparison can provide information regarding, for example, an increase or decrease in binding affinity between the mutant FBW7 protein and the FBW7 substrate in the presence of the candidate. For example, if the amount of luminescent light is greater than the amount of control luminescent light, and wherein the binding affinity between the mutant FBW7 protein and the FBW7 substrate is increased. As discussed further herein, such candidate compounds that increase a binding affinity between the mutant FWB7 peptide and the FBW7 substrate may be useful in, for example, the treatment of FWB7-mediated malignancies, such as in the methods of the present disclosure. In an embodiment, process block 1315 is optional.

### Kits for Performing Screening Assays

In another aspect, the present disclosure provides kits for performing screening assays, such as assays for characterizing an ability of a candidate compound to modulate a binding affinity between a mutant FBW7 protein and an FBW7 substrate. In an embodiment, the kits of the present disclosure are suitable to perform the screening assays of the present disclosure.

In an embodiment, the kits generally include a donor, an acceptor, a mutant FBW7 protein associated with either the donor or the acceptor, and an FWB7 substrate associated with the other of the donor bead or the acceptor bead. In an embodiment, the kit comprises a donor bead, an acceptor bead, a mutant FBW7 protein coupled to either the donor bead or the acceptor bead, and an FWB7 substrate coupled to the other of the donor bead or the acceptor bead.

As discussed further herein with respect to the methods of the present disclosure, the mutant FBW7 is coupled to either the donor bead or the acceptor bead. Likewise the FBW7 substrate is coupled to the other of the donor bead or the acceptor bead. In other words, the FBW7 substrate is coupled to whichever bead the mutant FBW7 protein is not coupled. Accordingly, in an embodiment, the FBW7 substrate is coupled to a surface of the donor bead; and wherein the mutant FBW7 protein is coupled to a surface of the acceptor bead. In another embodiment, the FBW7 substrate is coupled to a surface of the acceptor bead; and wherein the mutant FBW7 protein is coupled to surface of the donor bead.

In an embodiment, the FBW7 substrate and/or the mutant FBW7 protein are coupled to a surface of the donor bead or the acceptor bead. In an embodiment, such coupling includes a covalent bond. In an embodiment, the coupling includes a non-covalent interaction. As discussed further herein with respect to the methods of the present disclosure, in an embodiment, the FBW7 substrate and/or the mutant FBW7 protein include a polyhistadine tag, such as a terminal hexahistadine tag and a surface of the donor bead or the acceptor bead includes one or more metal ions configured to coordinate with the polyhistadine tag. In an embodiment, the FBW7 substrate and/or the mutant FBW7 protein include a biotin moiety and the donor or the acceptor beads include an avidin moiety, such as a streptavidin moiety.

As discussed further herein with respect to the methods of the present disclosure, the mutant FBW7 protein of the present aspect includes an FBW7 substrate-binding interface and or one or more mutations from a wildtype sequence. In an embodiment, the mutant FBW7 protein includes a missense mutation in an FBW7 substrate-binding interface that reduces the binding affinity of mutant FBW7 protein for the FBW7 substrate relative to a binding affinity of a wildtype FBW7 protein for the FBW7 substrate. In an embodiment, the mutant FBW7 protein includes a mutation selected from the group consisting of R465, R479, R505, R689, and combinations thereof relative to a wild type FBW7 protein, such as according to SEQ ID NO. 1.

As discussed further herein with respect to the methods of the present disclosure, the FBW7 substrate of the present aspect can be any substrate upon which an FBW7 protein, such as a mutant FBW7 protein, acts. In an embodiment, the FBW7 substrate is selected from the group consisting of c-Myc, n-Myc, Notch, cyclin E, c-Jun, PGC-1α, SREBP1, SREBP2, MCL1, MED13/13L, KLF5, KLF2, C/EBPδ, TGIF1, GATA2, GATA3, KLG10, KLF11, and C/EBPalpha. In an embodiment, the FBW7 substrate is selected from the group consisting of SEQ ID NOS. 2-20. In an embodiment, the FBW7 substrate is cyclin E.

In an embodiment, the donor bead includes a sensitizer configured to generate a reactive oxygen species when in an excited state. In an embodiment, the sensitizer is a photosensitizer configured to generate the reactive oxygen species when illuminated with stimulation electromagnetic radiation. In an embodiment, the reactive oxygen species is selected from the group consisting of peroxides, superoxide, hydroxyl radical, singlet oxygen, alpha-oxygen, and combinations thereof. In an embodiment, the photosensitizer is phthalocyanine. In an embodiment, the reactive oxygen species is singlet oxygen. In an embodiment, the sensitizer is disposed on a surface of the donor bead. In an embodiment, the sensitizer is distributed within the donor bead.

In an embodiment, the acceptor bead includes a luminescent compound configured to generate luminescent light when in proximity to the reactive oxygen species. In an embodiment, the luminescent compound is selected from thioxene, anthracene, rubrenein, and combinations thereof. In an embodiment, the luminescent compound is disposed on a surface of the bead. In an embodiment, the luminescent compound is distributed within a bead material.

In an embodiment, the acceptor bead is configured to be in proximity to the donor bead when coupled together by a candidate compound such that the acceptor bead is configured to generate luminescent light in response to contact and reaction with the reactive oxygen species generated by the donor bead. In an embodiment, the acceptor bead is configured to be a distance from the donor bead that is within a diffusion length of a reactive oxygen species half-life. In that regard, an acceptor bead may be configured to be within 200 nm of the donor bead when coupled by a candidate compound, such as where the donor bead is configured to generate singlet oxygen in response to illumination with stimulation electromagnetic radiation.

As above, the acceptors and donor described herein can have the form of a bead. In an embodiment, the beads include particulate materials such as glass beads, polymeric beads, metallic particles, semiconducting particles, liquid particles, and the like. In an embodiment, a bead size is suitable for making colloidal suspensions of the beads. Accordingly, in an embodiment, the beads have an average diameter in a range of about 100 nm to about 1,000 nm. In an embodiment, the beads have an average diameter in a range of about 520 nm to about 620 nm.

In an embodiment, the kit of the present disclosure includes control reagents for performing a control assay. As discussed further herein with respect to the methods of the present disclosure, the control reagents may be suitable for generating control luminescent light with which to compare luminescent light generated by candidate compounds.

In an embodiment, the control reagents include a control donor bead comprising a sensitizer configured to generate a control reactive oxygen species when in an excited state; a control acceptor bead comprising a control luminescent compound configured to generate luminescent light when in proximity to the control reactive oxygen species; an FBW7 protein coupled to either a surface of the control donor bead or a surface of the control acceptor bead; and an FBW7 substrate coupled to the other of a surface of the control donor bead or a surface of the control acceptor bead. In an embodiment, the FBW7 protein is a wildtype FBW7 protein. In an embodiment, the FBW7 protein is a mutant FBW7 protein. In an embodiment, the control reagents further include a non-specific assay agonist.

In an embodiment, the kit further includes a plate including a plurality of wells, wherein each of the plurality of wells is configured to hold the acceptor bead, the donor bead, and the candidate compound. In an embodiment, the plate is in a multiwell format such as a 48-well plate, a 96-well plate, a 384-well plate, a 1536-well plate, and the like. In this regard, the kit may be suitable to assay a number of candidate compounds, such as by high-throughput assays assaying several hundred or several thousand compounds.

In an embodiment, the kit further includes instructions, such as instructions for using reagents, such as including control reagents, for performing the assays described herein. In an embodiment, the instructions include description of conditions for incubating the candidate compound, such as reagent concentrations, medium pH, incubation temperature, incubation times, and the like. In an embodiment, the instructions include description of how to excite the donor to generate the reactive oxygen species, such as description regarding illumination wavelengths, light intensities, and the like. In an embodiment, the instructions include description regarding how to measure illumination light generated by the acceptor, such as with a photodetector, spectrophotometer, one or more filters, and the like. In an embodiment, the instructions include description of a comparison step for determining whether a binding affinity between a mutant FBW7 protein and an FBW7 substrate is indicated.

In an embodiment, the instructions include instructions for performing a method comprising: incubating the candidate compound with a donor configured to generate a reactive oxygen species when in an excited state and with an acceptor configured to generate luminescent light when the acceptor is in proximity to the reactive oxygen species; wherein either the donor or the acceptor is coupled to the mutant FBW7 protein and the other of the acceptor or the donor is coupled to the FBW7 substrate; exciting the donor to generate the reactive oxygen species; and measuring an amount of luminescent light generated by the acceptor in response to the reactive oxygen species, wherein if the amount of luminescent light is different from an amount of control luminescent light, a modulated binding affinity between the mutant FBW7 protein and the FBW7 substrate is indicated.

In an embodiment, the instructions include further description for one or more control assays. In an embodiment, the control assay instructions include a method including incubating a non-specific assay agonist with a control donor configured to generate a control reactive oxygen species when in an exited state and with a control acceptor configured to generate control luminescent light when the control acceptor is in proximity to the control reactive oxygen species; wherein either the control donor or the control acceptor is coupled to a mutant FBW7 protein and the other of the control acceptor or the control donor is coupled to an FBW7 substrate; exciting the control donor to generate the control reactive oxygen species; and measuring the amount of control luminescent light generated by the control acceptor in response to the control reactive oxygen species.

In an embodiment, the control assay instructions include a method including incubating the candidate compound with a control donor configured to generate a control reactive oxygen species when in an exited state and with a control acceptor configured to generate control luminescent light when the control acceptor is in proximity to the control reactive oxygen species; wherein either the control donor or the control acceptor is coupled to a wild type FBW7 protein and the other of the control acceptor or the control donor is coupled to an FBW7 substrate; exciting the control donor to generate the control reactive oxygen species; and measuring the amount of control luminescent light generated by the control acceptor in response to the control reactive oxygen species.

In an embodiment, the kit further comprises one or mediums, such as one or more buffered solutions, in which to dissolve or distribute the donor and the acceptor. As discussed further herein with respect to the screening assays and Examples of the present disclosure, solutions or suspensions of the acceptor and the donor having a pH in a range of about 6.0 to about 8.0, such as in a range from about 6.5 to about 7.5, generally provide assay conditions generally resulting relatively high amounts of luminescent light in response to successful candidate compound-mediated coupling between the donor bead and the acceptor bead. In that regard, in an embodiment, the medium is buffered to have a pH in a range of about 6.0 to about 8.0, such as in a range from about 6.5 to about 7.5.

### EXAMPLES

### Example 1: Development of a High-Throughput Assay That Detects FBW7-Substrate Interactions

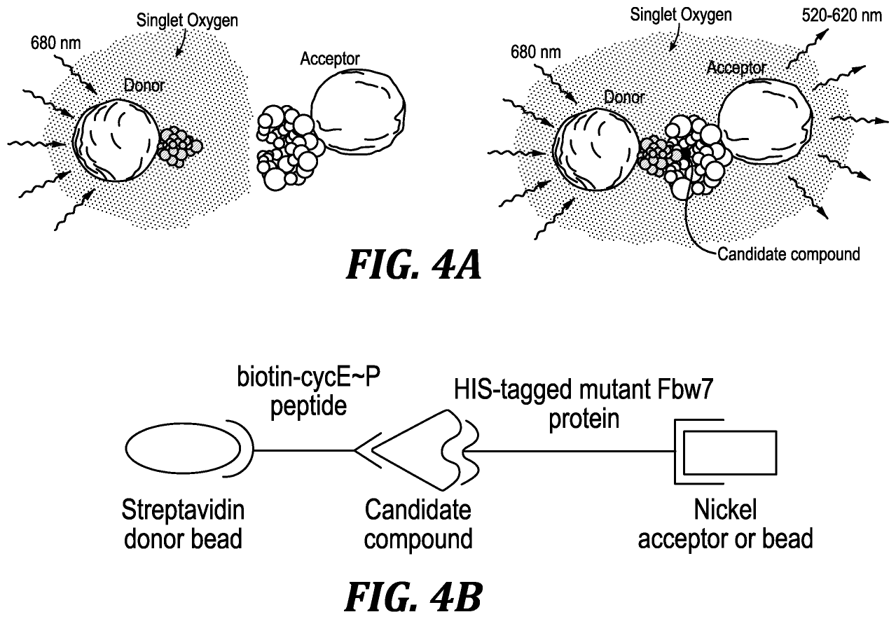

FBW7^{ARG} refers to a class of loss-of-function mutations of the of FBW7 ubiquitin ligase that target a key phosphate-binding arginine residue in the FBW7 substrate-binding domain. These mutations are commonly found in human cancers and promote tumorigenesis by impairing the interactions between FBW7 and its substrates, such as cyclin E and Myc. Described herein is a high throughput screen (HTS) to identify agonists, such as small molecules, that augment FBW7^{ARG}-substrate binding. In an embodiment, the HTS is based on the Amplified Luminescent Proximity Homogenous Assay (ALPHAscreen) because it detects protein-protein interactions with a large dynamic range. ALPHAscreen is a bead-based proximity assay wherein donor beads contain a photosensitizer, phthalocyanine, and acceptor beads contain thioxene, anthracene and rubrenein in sufficient quantities to generate a chemiluminescence/fluorescence reaction (FIG. 4A). Upon excitation with excitation illumination, such as light having a wavelength of 680 nm, of the donor beads, phthalocyanine converts ambient oxygen to singlet oxygen, which can diffuse approximately 200 nm within its 4-microsecond half-life. If an acceptor bead is in close proximity to an excited donor bead, singlet oxygen transfers its energy to the acceptor, resulting in emitted light at a broad bandwidth of ˜520-620 nm. The HTS-compatible FBW7/cyclin E ALPHAscreen assay described herein uses streptavidin coupled donor beads and Ni-chelate acceptor beads. In this assay, biotinylated cyclin E phosphopeptide and the His-tagged FBW7 mutant protein have high affinity for the donor and acceptor beads, respectively (FIG. 4B). A small molecule that is able to bridge the binding sites of the peptide and the mutant protein (or induce a conformational change that increases affinity of the protein for the peptide) will bring the donor and acceptor beads in close proximity to each other, resulting in an ALPHA signal. As designed, this assay format is a very sensitive, non-radioactive, homogenous “mix-and-read” format that is HT compatible in 384 and 1536 well formats.

### Example 2: Purification of Dimeric Skp1-FBW7

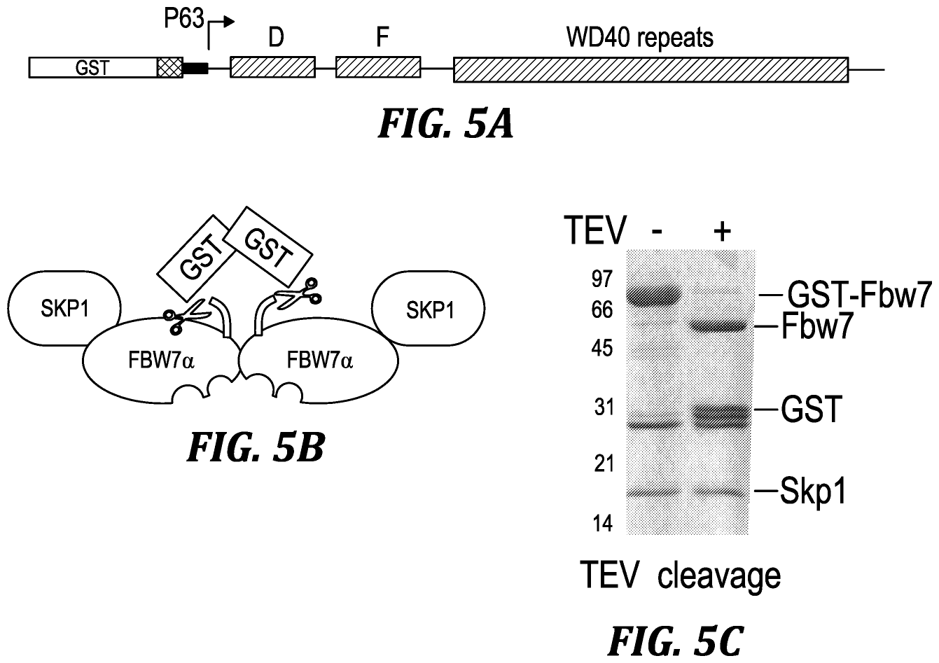

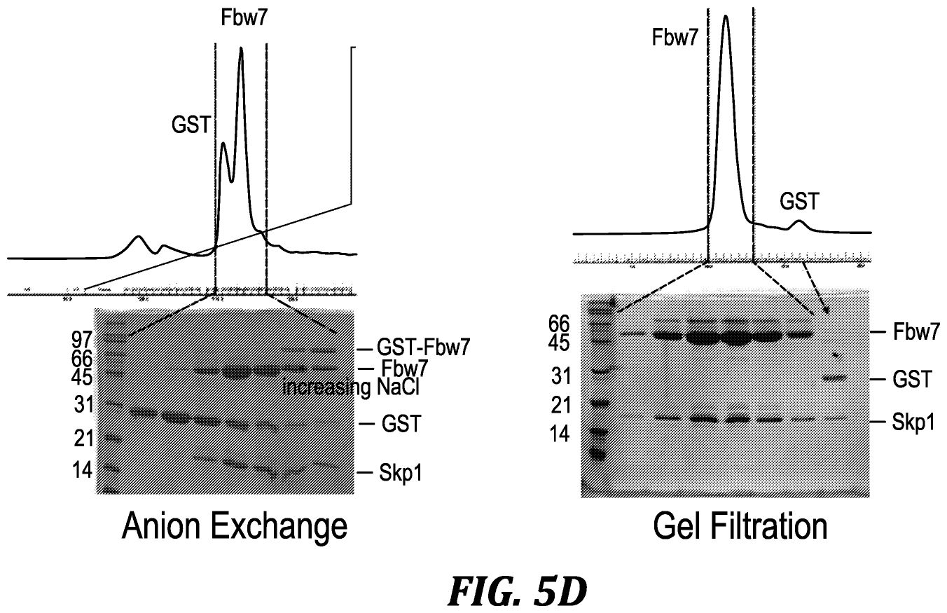

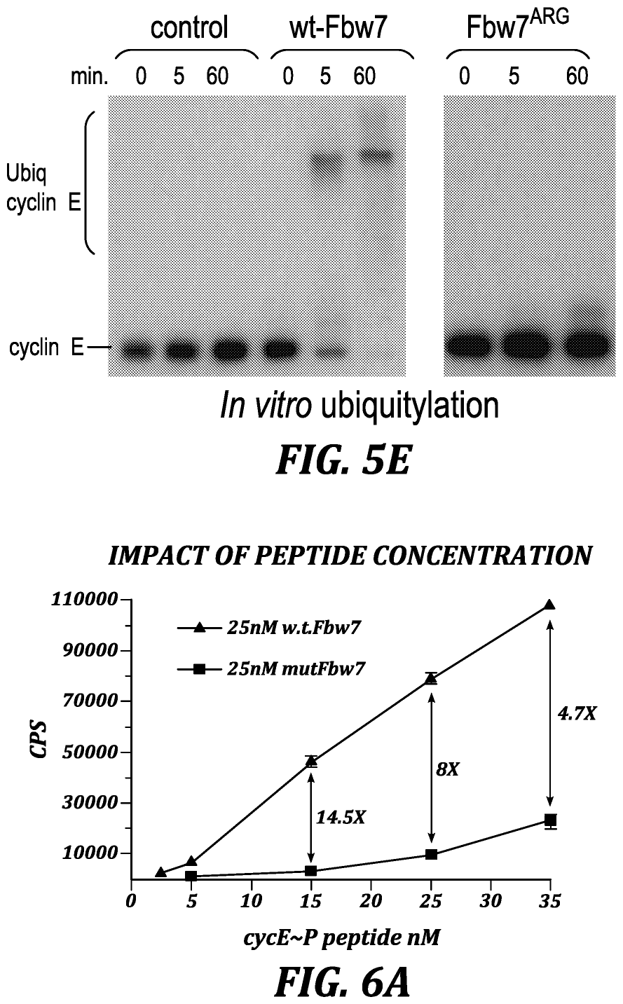

Because FBW7 is dimeric in vivo, the HTS is based upon dimeric FBW7. However, this had proved difficult for other groups, and resulted in structures being derived from FBW7 monomers. Methods were, thus, first developed to obtain highly purified FBW7 proteins that are active and soluble. We first tested a series of FBW7 N-terminal truncation mutants that were predicted to be soluble and found that a construct beginning at P63 was soluble. We constructed a baculovirus vector expressing GST-P63-FBW7 with a TEV protease site and a flexible linker between the GST and FBW7 moieties that greatly improved TEV cleavage (FIGS. 5A, 5B). We co-infected SF9 insect cells with viruses expressing FBW7 and Skp1 (Skp1 binding improves FBW7 solubility and all endogenous FBW7 is bound to Skp1 in vivo). GST-FBW7 from cell lysates was bound to GST-resin and eluted with glutathione. GST-TEV-FBW7 was cleaved with TEV to remove the GST tag (FIG. 5C) and purified by anion exchange chromatography (SourceQ column-GE) and size exclusion chromatography (Superdex200-GE) (FIGS. 5D, 5E). This approach yields about 8 mg of homogeneous FBW7 protein/24 liters of SF9 culture. We purified wt-FBW7 and the FBW7^{ARG}. In our reconstituted in vitro Cyclin E polyubiquitylation assay, wt-FBW7 was highly active and FBW7^{ARG} was inactive (FIG. 5E).

### Example 3: Development of 384- and 1536-Well ALPHAscreens

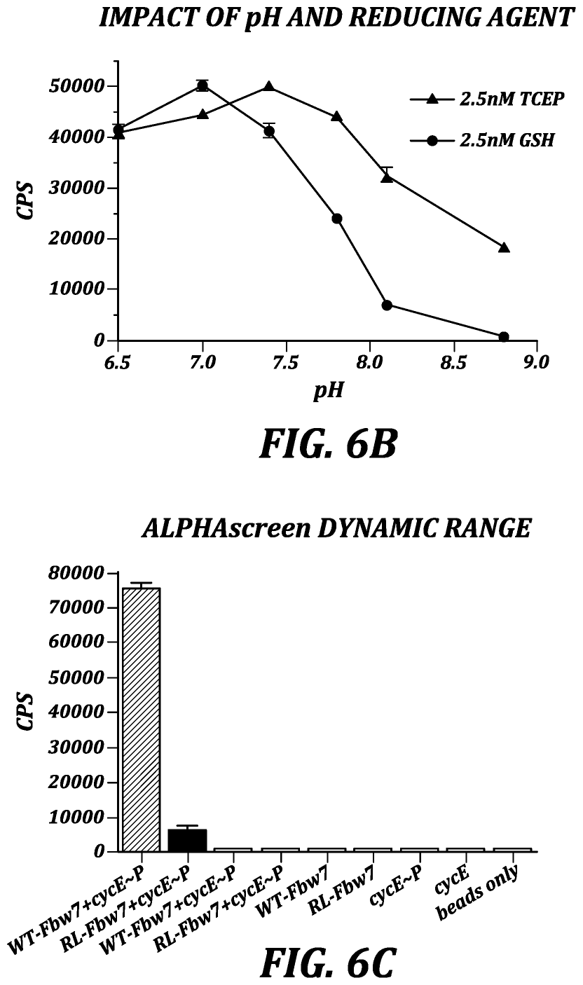

Studies in 384-well format were performed using the ENVISION system. Reagent concentrations (see FIG. 6A), order of addition, pH (see FIG. 6B), and reducing agent were altered with the goal of enhancing the dynamic range between wt-FBW7 (the positive control) and FBW7^{ARG} (FIG. 6 and not shown). Wt-FBW7 produced a signal 100× greater with phosphorylated cyclin E degron peptide compared with unphosphorylated, which represents the assay's maximum range (FIG. 6C). Remarkably, although inactive in ubiquitylation assays, FBW7^{ARG} produces an ALPHA signal with phosphorylated cyclin E peptide that is intermediate to that obtained with wt-FBW7 and phosphorylated or unphosphorylated cyclin E peptides. This assay thus detects the residual affinity of the FBW7^{ARG}-substrate interaction, which, as predicted, is stronger than wt-FBW7 protein with unphosphorylated cyclin E peptide. Concurrent with this primary assay, counter ALPHAscreens were developed which omitted either FBW7 or peptide (to eliminate hits that are general ALPHAscreen agonists), or used an irrelevant phosphopeptide (to eliminate compounds that simply augment binding to phosphopeptides).

### Example 4: Performance Characteristics of the Optimized 384-Well FBW7 Screening Assay

In the 384-well screening assay, 10 uL of acceptor beads with 25 nM of the FBW7^{ARG} (RLFBW7) are dispensed to test wells; acceptor beads with 25 nM of the wt-FBW7 protein (WTFBW7) are dispensed to control wells. Test compounds are then added by pintool transfer and the mixture is incubated for 30 minutes. An additional 10 uL of donor beads with 25 nM cyclin E phosphopeptide is then added to all wells and the plate is incubated for 4 hours before the ALPHA signal is measured.

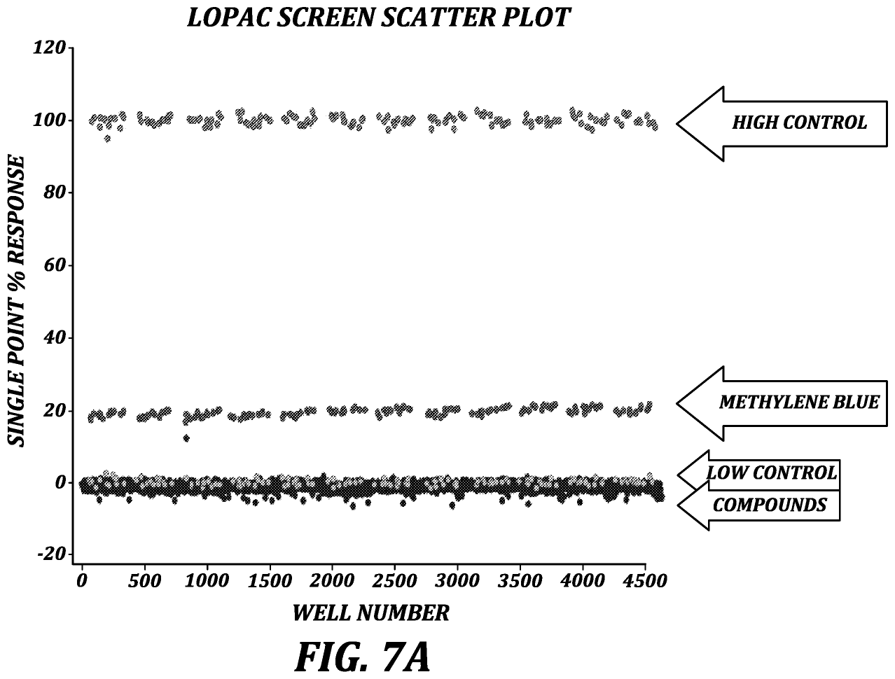

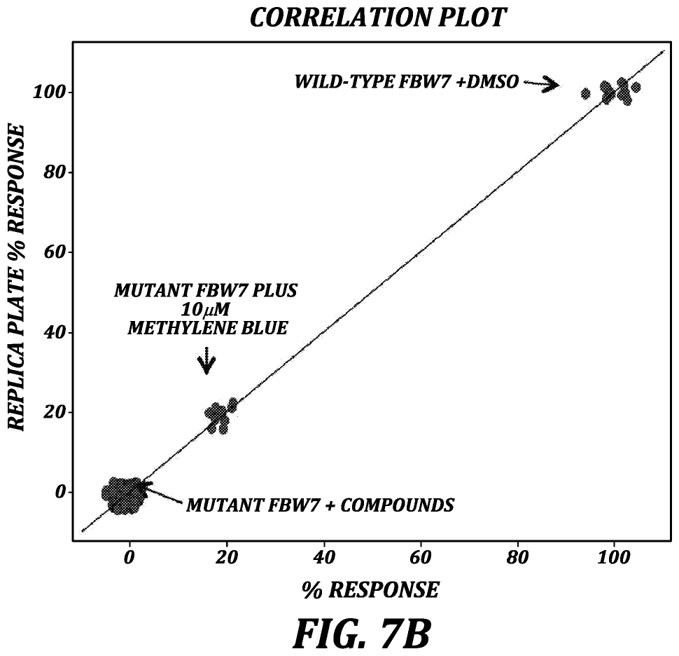

To determine the performance of optimized FBW7 assay in terms of robustness (S:B, Z′) under HTS conditions, the assay was screened against the Sigma LOPAC (Library of Pharmacologically Active Compounds). LOPAC compounds were tested in triplicate at a single concentration of 12.5 μm (0.5% DMSO final) using wildtype FBW7 and Methylene blue (an FBW7 independent non-specific assay agonist) as positive controls. Each plate contained high (wt-FBW7) and low (mutant FBW7) signal control wells, which were used in Z′ factor calculations. An activity scatterplot of all compounds tested, as well as positive and negative controls, is shown in FIG. 7A. As indicated from the positive and negative control scatterplots, the entire LOPAC screen assay demonstrated a superlative Z′ factor (0.95±0.01) and an excellent S:B ratio of 14.6±2.3. Further, a scatterplot of replicate measurements yields an r²=0.998, indicating high reproducibility in assay data (FIG. 7B).

### Example 5: Assay Miniaturization to a 1536-Well Format

In preparation for large-scale screening, the assay was miniaturized to a 1536-well format. The miniaturized screen performed outstandingly in all respects. The screen was enhanced with respect to several parameters and assayed its performance using mutant FBW7 and wt-FBW7, as well as non-specific assay agonists. We found that decreased FBW7 concentration was well tolerated, that the assay's performance was outstanding at [FBW7]>12.nM. For example, the following Z′-scores/FBW7 concentrations were obtained: 6.25 nM/Z′=0.62, 12.5 nm/Z′=0.85, 25.0 nM/Z′=0.84. The impact of a 4 hour versus a 20 hr incubation period was tested, and improved assay performance was observed with the overnight incubation prior to plate reading.

Enhanced conditions for the 1536-well format were as follows:

1. Add 2.5 uL of RLFBW7/cycE˜P/Ni beads: Final [25 nM/25 nM/10 ug/ml].

2. Add 2.5 uL of WTFBW7/cycE˜P/Ni beads: Final [25 nM/25 nM/10 ug/ml.

3. Centrifuge

4. Add 2.5 uL of SA beads to all wells Final [10 ug/ml],

5. Add 30 nL of compounds,

6. Read ALPHAscreen on Envision

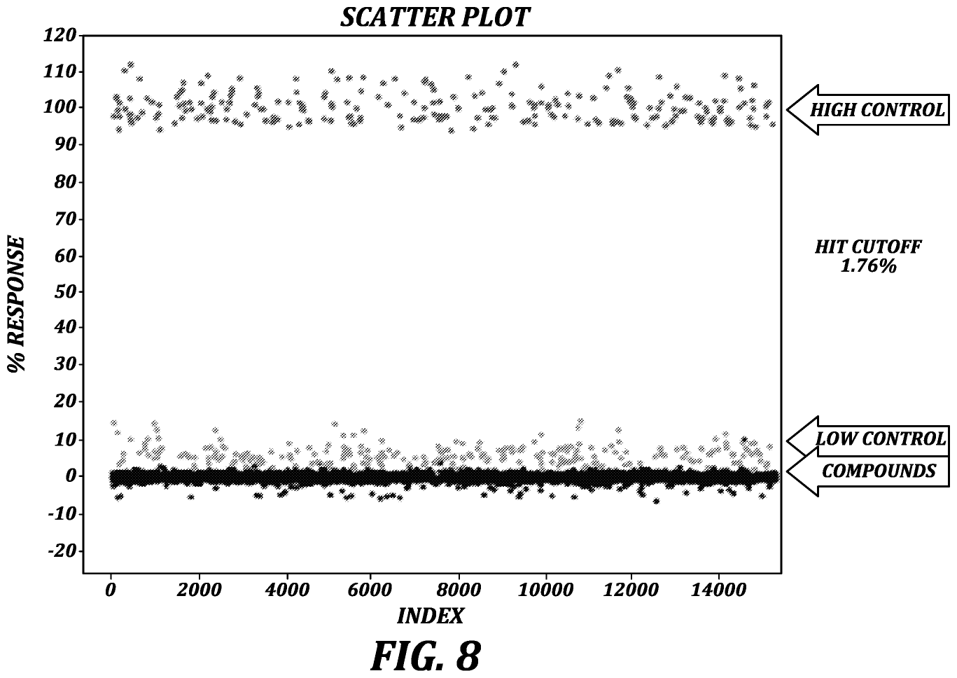

After assay miniaturization, a 10K pilot screen was performed to assess its performance. It was not expected to find strong hits in these limited pilots. The results of this pilot are shown in FIG. 8. 11,809 compounds were tested at ˜6 uM and obtained the following performance characteristics: ⋅Ave Z′=0.84±0.02, Ave Z=0.86±0.01, Ave S:B=8.14±1.23.

### Example 6: 645k Primary FBW7^{ARG}-Cyclin E Agonist Screen

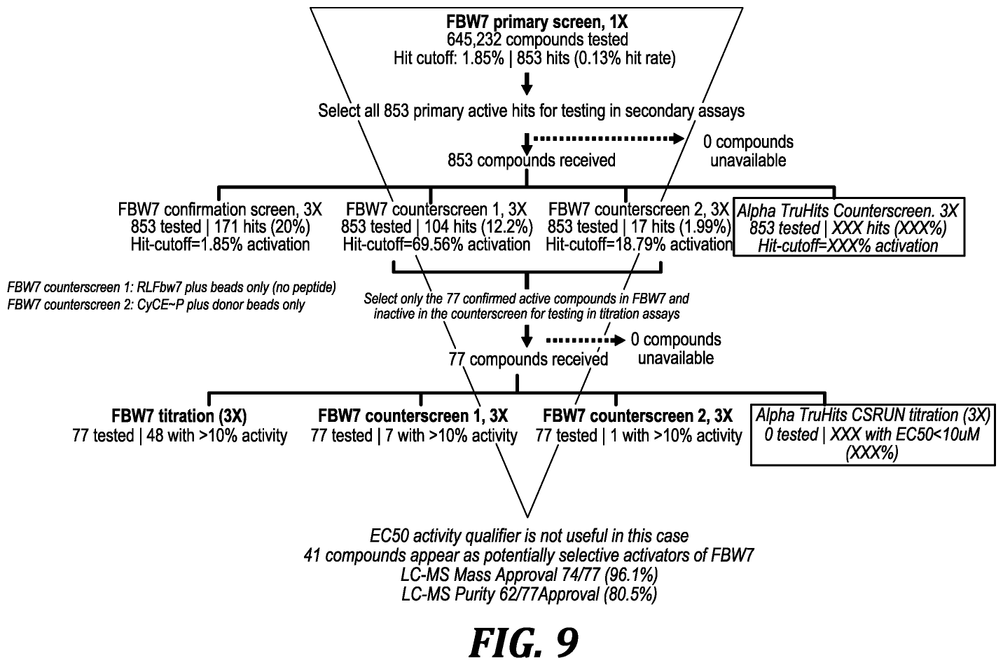

The large-scale FBW7 agonist ALPHAscreen was completed using FBW7^{ARG} and a phosphorylated cyclin E (pCycE) degron peptide. Wt-FBW7+pCycE-peptide was the “high control”, and Wt-FBW7+ unphosph. CycE peptide was the “low control”. The screen was performed on the 645,000-compound Scripps Diversity Library using 1536-well format and drug concentrations of 26.1 μm. The flowthrough of the entire screen and counter-screens is shown in FIG. 9. An algorithm aided hit identification based on the average percent activation for all compounds and their standard deviation. Active compounds were those that exhibited greater percent activation than the cutoff parameter (1.85%), which yielded 835 primary hits. The screen exhibited outstanding performance characteristics (Z′ of 0.88+/−0.03; signal-to-background (S:B) of 69.35+/−24.11; n=518 plates).

### Example 7: Hit Confirmation and Counter-Screens

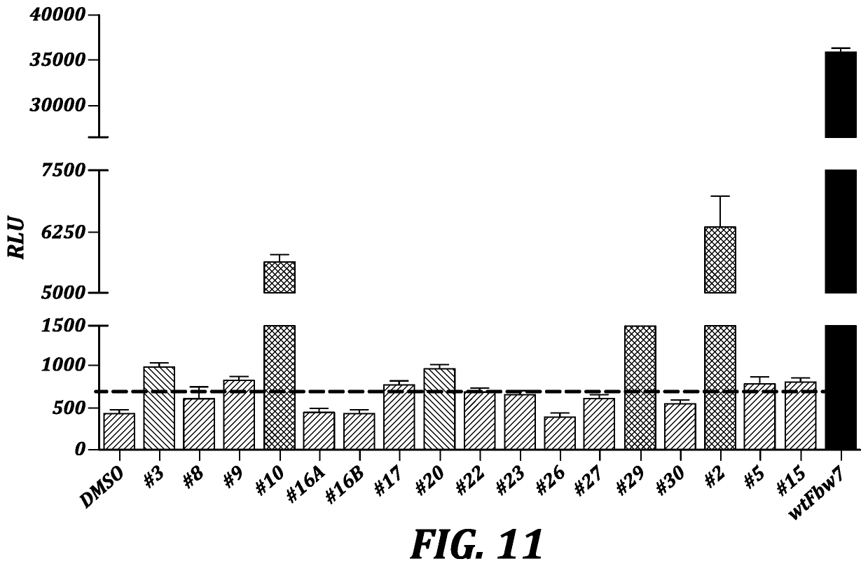

A confirmation assay of all 853 compounds in triplicate using a single concentration (26.1 μM). Assay performance was outstanding (Z′−0.93+/−0/01 and S:B 18.44+/−0.68, which allowed us to set the hit-cut-off at 1.85% activity, which was necessary to retain any remotely active compounds, and led to 171 confirmed hits. Based on the anticipated mechanism of action, attention was focused on those hits that exhibited specificity for the combination of both FBW7^{ARG} and cycE peptide (CRUN1) but not FBW7^{ARG} alone (CSRUN1) or cycE peptide alone (CSRUN2) (FIG. 11). As expected, the strongest hits proved to be non-specific assay agonists, and 77 compounds were selected for further studies.

### Example 8: Hit Titration/Dose-Response Strategy

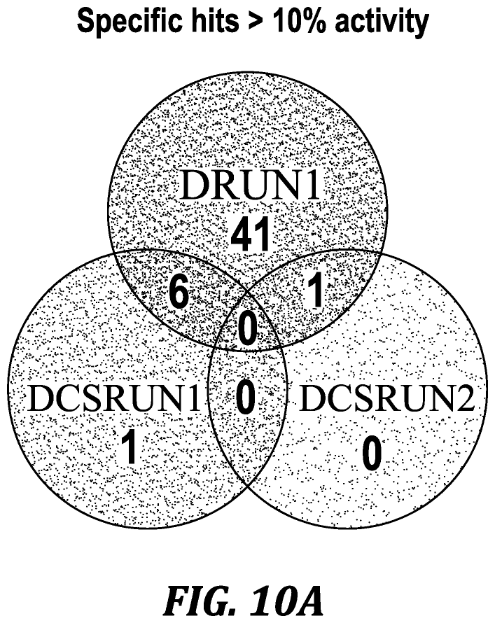

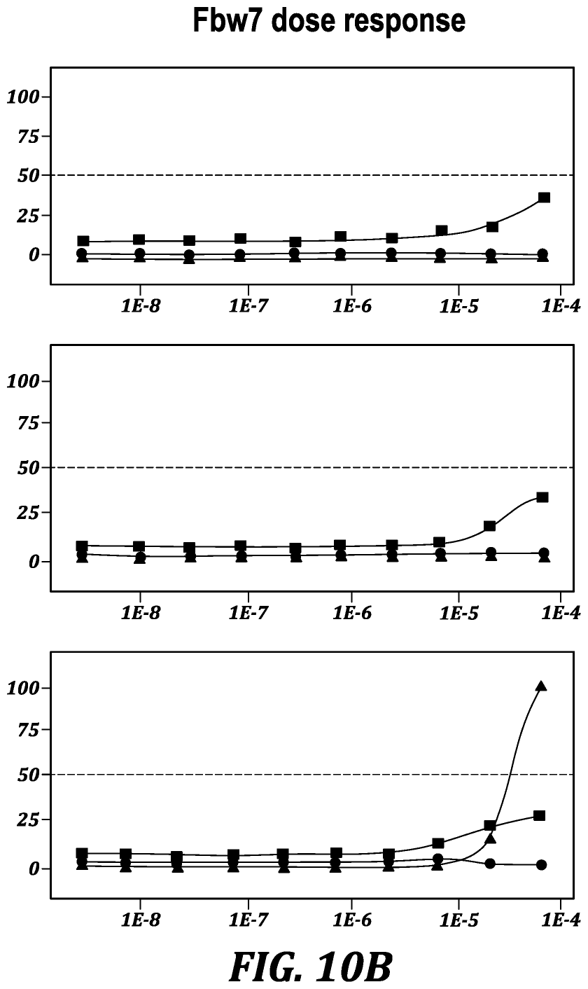

The 77 hits were subjected to a dose-response assay in which each compound was tested in triplicate in a 10 point 3-fold dilutional series, to a maximum concentration of approximately 87 μM. Three screens were performed, using FBW7^{ARG}+pCycE (DRUN), FBW7^{ARG} (DCSRUN1), or pCycE alone (DCSRUN2). See FIG. 10A. All of the assays performed extremely well, and threshold of 10% activation (relative to WT-FBW7+ pCycE) was set as positive. 41 compounds had an average maximum activity >10%, albeit at high concentrations, as expected (FIG. 10B). Many hits fell into shared structure cluster ID groups, reinforcing the idea that they function as specific agonists.

### Example 9: Hit Validation and Follow-Up Activity Assays

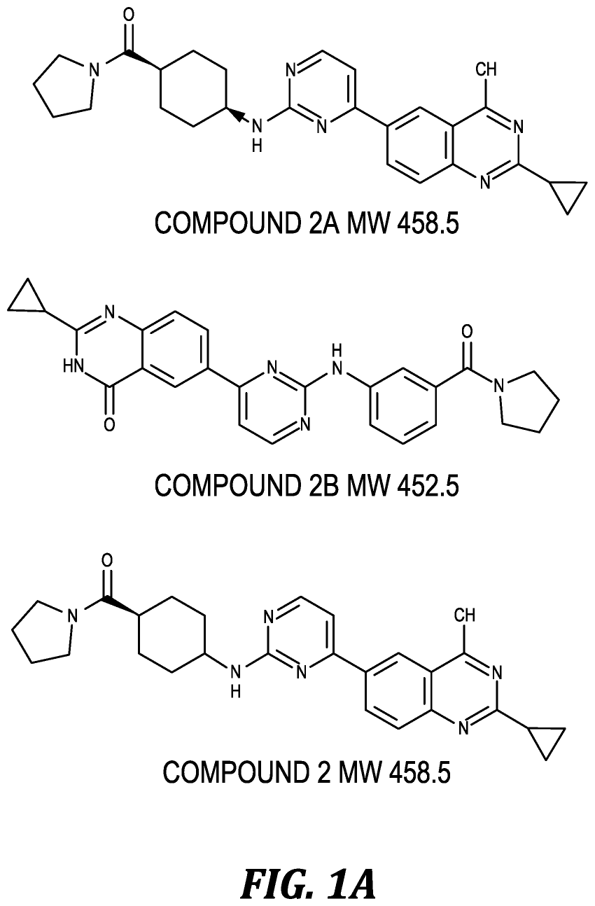

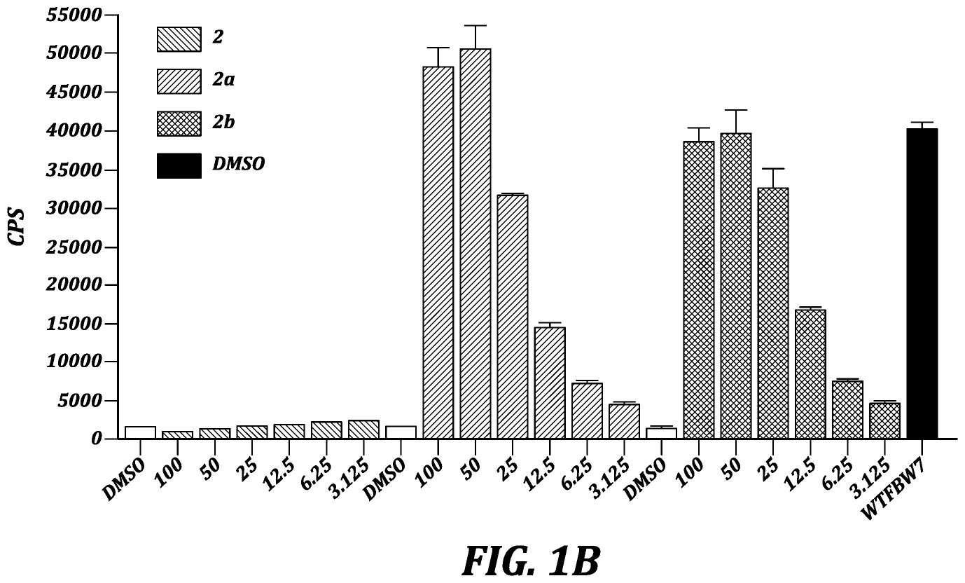

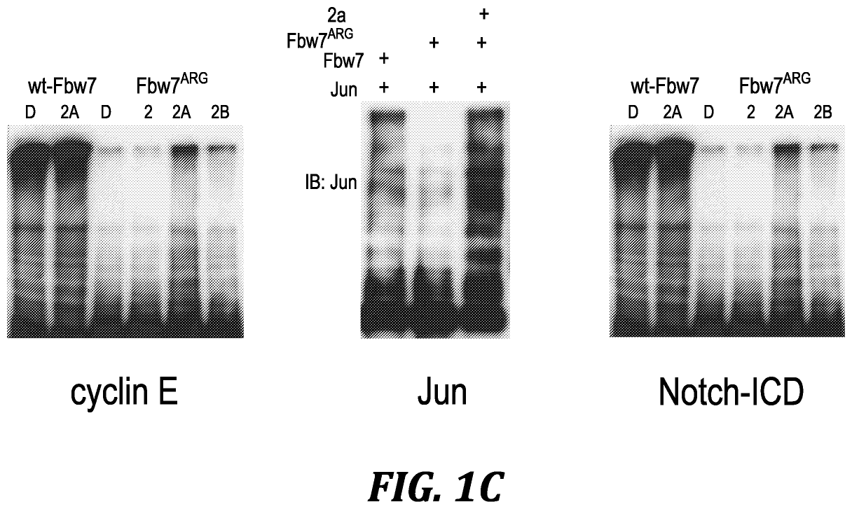

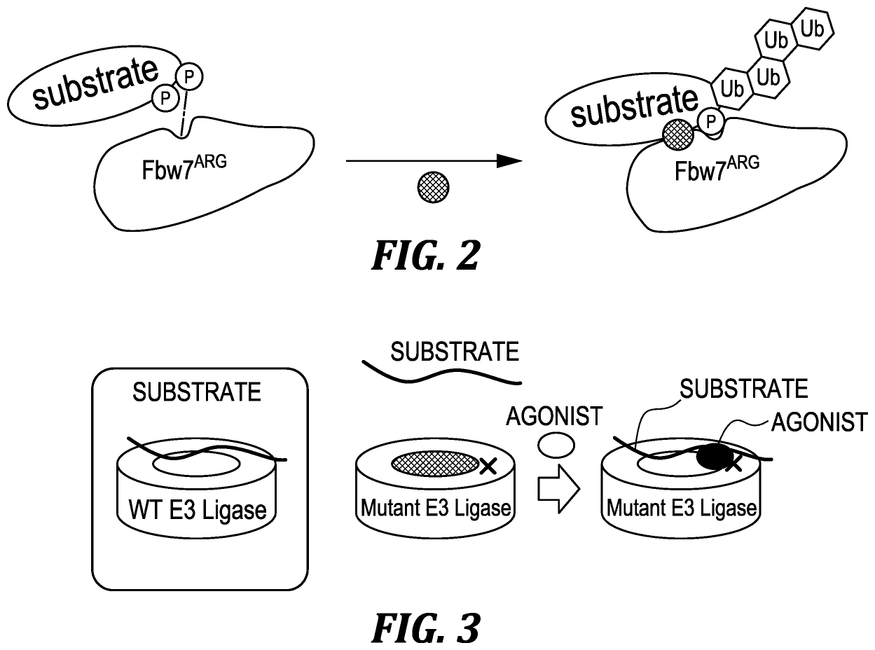

20 hits with favorable dose response and specificity were selected, which largely fell into fell into 5 structure clusters. All hits underwent LC-MS mass and LC-MS-purity testing. Although preliminary, very stringent ALPHAscreening conditions have confirmed that approximately ⅔ rds of these compounds are reproducing activity (see FIG. 1).

### Example 10: Lead Compounds with FBW7^{ARG} Agonist Activity

Thus far, the focus has been primarily on compounds based upon hit #2 (FIG. 11) and analogs in ubiquitylation assays and additional ALPHAscreens. As shown in FIG. 1, these compounds exhibit apparent stereospecificity in ubiquitylation and ALPHAscreen assays. Note, that while parent compound 2 was the original hit in the HTS, a new aliquot from a different lot appeared to be inactive in subsequent validation assays. In vitro ubiquitylation assays have been performed using full length cyclin E, c-Jun, Notch, and Myc. Despite being initially identified with the cyclin E peptide as substrate, compound 2A restores the ability of recombinant dimeric FBW7^{ARG} to direct ubiquitylation of each of these substrates, except for c-Myc (FIG. 1C and not shown). Due to limitations in chemical syntheses, a mechanism of action (e.g. molecular glue versus allosteric) or other features such as substrate specificity, in vivo activity, etc. have not been determined.

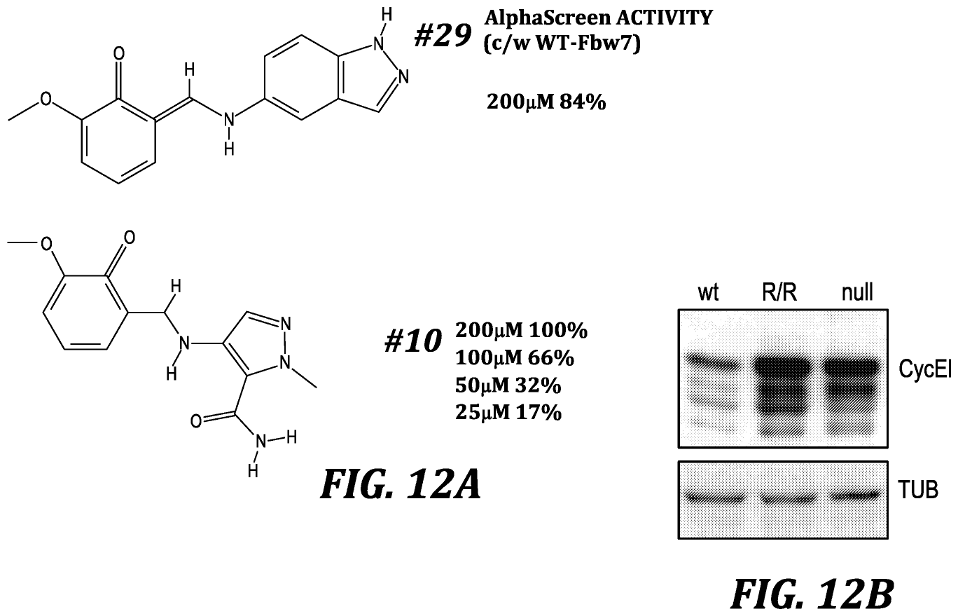

In addition to the compounds shown in FIG. 1, we have also begun to study other lead scaffolds identified in the primary screen (#10 & #29), which showed activity when retested that was specific for the combination of both FBW7 and substrate (FIG. 12A). Both compounds appear fairly reactive, and despite their specificity for FBW7-substarte combinations, it is possible they act covalently in the ALPHAscreen.

### Example 11: Physiologic Systems for Cell-Based Studies of FBW7 Agonists

We have developed a wide range of physiologic knock-in models to test candidate FBW7 agonists for activity in cell-based screens. These include human cells lines in which we have used either adeno-associated virus-based gene targeting, or more recently, CRISPR-Cas9. FIG. 12B shows an example of Hct116 colorectal carcinoma cells in which both endogenous FBW7 alleles were mutated to R505C. Note the stabilization of cyclin E in these cells, which provides a sensitive and highly physiologic assay for FBW7 agonists. Myc turnover is also prolonged to the same extent as in FBW7 null Hct116 cells (not shown).

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application. The term “about” means plus or minus 5% of the stated value.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for characterizing an ability of a candidate compound to modulate a binding affinity between a mutant F-box/WD repeat-containing protein 7 (FBW7) peptide and an FBW7 substrate, the method comprising:
incubating the candidate compound with a donor bead configured to generate a reactive oxygen species when in an excited state and with an acceptor bead configured to generate luminescent light when the acceptor bead is in proximity to the reactive oxygen species; wherein either the donor bead or the acceptor bead is coupled to the mutant FBW7 protein and the other of the acceptor bead or the donor bead is coupled to the FBW7 substrate;
exciting the donor bead to generate the reactive oxygen species; and
measuring an amount of luminescent light generated by the acceptor bead in response to the reactive oxygen species, wherein if the amount of luminescent light is different from an amount of control luminescent light, a modulated binding affinity between the mutant FBW7 protein and the FBW7 substrate is indicated.

2. The method of claim 1, wherein the donor bead comprises sensitizer configured to generate the reactive oxygen species when the sensitizer is in an excited state.

3. The method of claim 2, wherein the sensitizer is a photosensitizer configured to generate the reactive oxygen species when the sensitizer is illuminated with stimulation electromagnetic radiation.

4. The method of claim 3, wherein the photosensitizer is phthalocyanine.

5. The method of claim 1, wherein the acceptor bead comprises a luminescent compound configured to generate luminescent light when the luminescent compound is in proximity to the reactive oxygen species.

6. The method of claim 5, wherein the luminescent compound is selected from thioxene, anthracene, rubrenein, and combinations thereof.

7. The method of claim 1, wherein the reactive oxygen species is singlet oxygen.

8. The method of claim 1, wherein the FBW7 substrate is selected from the group consisting of c-Myc, n-Myc, Notch, cyclin E, c-Jun, PGC-1α, SREBP1, SREBP2, MCL1, MED13/13L, KLF5, KLF2, C/EBPδ, TGIF1, GATA2, GATA3, KLG10, KLF11, and C/EBPalpha according to SEQ ID NOS. 2-20, respectively.

9. The method of claim 1, wherein the FBW7 substrate is cyclin E according to SEQ ID NO. 5.

10. The method of claim 1, wherein the mutant FBW7 protein includes a missense mutation in a substrate binding interface that reduces the binding affinity of mutant FBW7 protein for the FBW7 substrate relative to a binding affinity of a wildtype FBW7 protein for the FBW7 substrate.

11. The method of claim 1, wherein the mutant FBW7 protein includes a mutation selected from the group consisting of R465, R479, R505, R689, and combinations thereof relative to a wildtype FBW7 protein according to SEQ ID NO. 1.

12. The method of claim 1, wherein the FBW7 substrate is coupled to a surface of the donor bead; and wherein the mutant FBW7 protein is coupled to a surface of the acceptor bead.

13. The method of claim 1, wherein the FBW7 substrate is coupled to a surface of the acceptor bead; and wherein the mutant FBW7 protein is coupled to a surface of the donor bead.

14. The method of claim 1, wherein incubation includes:
contacting the candidate compound with the acceptor bead for an acceptor-bead incubation time in a range of about 10 minutes to about one hour; and
contacting the candidate compound with the donor bead for a donor-bead incubation time in a range of about 4 hours to about 20 hours.

15. The method of claim 1, wherein the amount of control luminescent light is generated by a control assay.

16. The method of claim 15, wherein control assay comprises:
incubating a non-specific assay agonist with a control donor bead configured to generate a control reactive oxygen species when in an exited state and with a control acceptor bead configured to generate control luminescent light when the control acceptor bead is in proximity to the control reactive oxygen species; wherein either the control donor bead or the control acceptor bead is coupled to a mutant FBW7 protein and the other of the control acceptor bead or the control donor bead is coupled to an FBW7 substrate;
exciting the control donor bead to generate the control reactive oxygen species; and
measuring the amount of control luminescent light generated by the control acceptor bead in response to the control reactive oxygen species.

17. The method of claim 16, wherein the non-specific assay agonist is methylene blue.

18. The method of claim 15, wherein control assay comprises:
incubating the candidate compound with a control donor bead configured to generate a control reactive oxygen species when in an exited state and with a control acceptor bead configured to generate control luminescent light when the control acceptor bead is in proximity to the control reactive oxygen species; wherein either the control donor bead or the control acceptor bead is coupled to a wild type FBW7 protein and the other of the control acceptor bead or the control donor bead is coupled to an FBW7 substrate;
exciting the control donor bead to generate the control reactive oxygen species; and
measuring the amount of control luminescent light generated by the control acceptor bead in response to the control reactive oxygen species.

19. The method of claim 18, wherein the FBW7 substrate is unphosphorylated cyclin E.

20. The method of claim 1, wherein the amount of luminescent light is greater than the amount of control luminescent light, and wherein the binding affinity between the mutant FBW7 protein and the FBW7 substrate is increased.

21. The method of claim 1, further comprising comparing the amount of luminescent light generated by the acceptor bead in response to the reactive oxygen species and the amount of control luminescent light generated by the control acceptor bead in response to the control reactive oxygen species.

22. A kit comprising:
a donor bead comprising a sensitizer configured to generate a reactive oxygen species when in an excited state;
an acceptor bead comprising a luminescent compound configured to generate luminescent light when in proximity to the reactive oxygen species;
a mutant FBW7 protein coupled to either a surface of the donor bead or a surface of the acceptor bead; and
an FBW7 substrate coupled to the other of a surface of the donor bead or a surface of the acceptor bead.

23. The kit of claim 22, wherein the sensitizer is a photosensitizer configured to generate the reactive oxygen species when illuminated with stimulation electromagnetic radiation.

24. The kit of claim 22, wherein the photosensitizer is phthalocyanine.

25. The kit of claim 22, wherein the luminescent compound is selected from thioxene, anthracene, rubrenein, and combinations thereof

26. The kit of claim 22, wherein the reactive oxygen species is singlet oxygen.

27. The kit of claim 22, wherein the FBW7 substrate is coupled to a surface of the donor bead; and wherein the mutant FBW7 protein is coupled to a surface of the acceptor bead.

28. The kit of claim 22, wherein the FBW7 substrate is coupled to a surface of the acceptor bead; and wherein the mutant FBW7 protein is coupled to surface of the donor bead.

29. The kit of claim 22, wherein the FBW7 substrate is selected from the group consisting of c-Myc, n-Myc, Notch, cyclin E, c-Jun, PGC-1α, SREBP1, SREBP2, MCL1, MED13/13L, KLF5, KLF2, C/EBPδ, TGIF1, GATA2, GATA3, KLG10, KLF11, and C/EBPalpha according to SEQ ID NOS 2-20, respectively.

30. The kit of claim 22, wherein the FBW7 substrate is cyclin E according to SEQ ID NO. 5.

31. The kit of claim 22, wherein the mutant FBW7 protein includes a missense mutation in a substrate binding interface that reduces the binding affinity of mutant FBW7 protein for the FBW7 substrate relative to a binding affinity of a wildtype FBW7 protein for the FBW7 substrate.

32. The kit of claim 22, wherein the mutant FBW7 protein includes a mutation selected from the group consisting of R465, R479, R505, R689, and combinations thereof relative to a wildtype FBW7 protein according to SEQ ID NO. 1.

33. The kit of claim 22, further comprising:
a control donor bead comprising a sensitizer configured to generate a control reactive oxygen species when in an excited state;
a control acceptor bead comprising a control luminescent compound configured to generate luminescent light when in proximity to the control reactive oxygen species;
an FBW7 protein coupled to either a surface of the control donor bead or a surface of the control acceptor bead; and
an FBW7 substrate coupled to the other of a surface of the control donor bead or a surface of the control acceptor bead.

34. The kit of claim 33, wherein the FBW7 protein is a wildtype FBW7 protein according to SEQ ID NO. 1.

35. The kit of claim 33, further comprising a non-specific assay agonist.

36. The kit of claim 33, wherein the FBW7 protein is a mutant FBW7 protein.

37. The kit of claim 22, further comprising a plate including a plurality of wells, wherein each of the plurality of wells is configured to hold the acceptor bead, the donor bead, and the candidate compound.


SEQUENCE LISTING (20 sequences)

SEQ ID NO: 1 - protein, 707 aa, Homo sapiens
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 2 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 3 - protein, 464 aa, Homo sapiens
MPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 4 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 5 - protein, 410 aa, Homo sapiens
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 6 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 7 - protein, 797 aa, Mus musculus
MAWDMCSQDSVWSDIECAALVGEDQPLCPDLPELDLSELDVNDLDTDSFLGGLKWCSDQSEIISNQYNNEPANIFEKIDEENEANLLAVLTETLDSLPVDEDGLPSFDALTDGAVTTDNEASPSSMPDGTPPPQEAEEPSLLKKLLLAPANTQLSYNECSGLSTQNHAANHTHRIRTNPAIVKTENSWSNKAKSICQQQKPQRRPCSELLKYLTTNDDPPHTKPTENRNSSRDKCASKKKSHTQPQSQHAQAKPTTLSLPLTPESPNDPKGSPFENKTIERTLSVELSGTAGLTPPTTPPHKANQDNPFKASPKLKPSCKTVVPPPTKRARYSECSGTQGSHSTKKGPEQSELYAQLSKSSGLSRGHEERKTKRPSLRLFGDHDYCQSLNSKTDILINISQELQDSRQLDFKDASCDWQGHICSSTDSGQCYLRETLEASKQVSPCSTRKQLQDQEIRAELNKHFGHPCQAVFDDKSDKTSELRDGDFSNEQFSKLPVFINSGLAMDGLFDDSEDESDKLSYPWDGTQPYSLFDVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEAYEHERLKRDEYRKEHEKRESERAKQRERQKQKAIEERRVIYVGKIRPDTTRTELRDRFEVFGEIEECTVNLRDDGDSYGFITYRYTCDAFAALENGYTLRRSNETDFELYFCGRKQFFKSNYADLDTNSDDFDPASTKSKYDSLDFDSLLKEAQRSLRR

SEQ ID NO: 8 - protein, 1147 aa, Homo sapiens
MDEPPFSEAALEQALGEPCDLDAALLTDIEDMLQLINNQDSDFPGLFDPPYAGSGAGGTDPASPDTSSPGSLSPPPATLSSSLEAFLSGPQAAPSPLSPPQPAPTPLKMYPSMPAFSPGPGIKEESVPLSILQTPTPQPLPGALLPQSFPAPAPPQFSSTPVLGYPSPPGGFSTGSPPGNTQQPLPGLPLASPPGVPPVSLHTQVQSVVPQQLLTVTAAPTAAPVTTTVTSQIQQVPVLLQPHFIKADSLLLTAMKTDGATVKAAGLSPLVSGTTVQTGPLPTLVSGGTILATVPLVVDAEKLPINRLAAGSKAPASAQSRGEKRTAHNAIEKRYRSSINDKIIELKDLVVGTEAKLNKSAVLRKAIDYIRFLQHSNQKLKQENLSLRTAVHKSKSLKDLVSACGSGGNTDVLMEGVKTEVEDTLTPPPSDAGSPFQSSPLSLGSRGSGSGGSGSDSEPDSPVFEDSKAKPEQRPSLHSRGMLDRSRLALCTLVFLCLSCNPLASLLGARGLPSPSDTTSVYHSPGRNVLGTESRDGPGWAQWLLPPVVWLLNGLLVLVSLVLLFVYGEPVTRPHSGPAVYFWRHRKQADLDLARGDFAQAAQQLWLALRALGRPLPTSHLDLACSLLWNLIRHLLQRLWVGRWLAGRAGGLQQDCALRVDASASARDAALVYHKLHQLHTMGKHTGGHLTATNLALSALNLAECAGDAVSVATLAEIYVAAALRVKTSLPRALHFLTRFFLSSARQACLAQSGSVPPAMQWLCHPVGHRFFVDGDWSVLSTPWESLYSLAGNPVDPLAQVTQLFREHLLERALNCVTQPNPSPGSADGDKEFSDALGYLQLLNSCSDAAGAPAYSFSISSSMATTTGVDPVAKWWASLTAVVIHWLRRDEEAAERLCPLVEHLPRVLQESERPLPRAALHSFKAARALLGCAKAESGPASLTICEKASGYLQDSLATTPASSSIDKAVQLFLCDLLLVVRTSLWRQQQPPAPAPAAQGTSSRPQASALELRGFQRDLSSLRRLAQSFRPAMRRVFLHEATARLMAGASPTRTHQLLDRSLRRRAGPGGKGGAVAELEPRPTRREHAEALLLASCYLPPGFLSAPGQRVGMLAEAARTLEKLGDRRLLHDCQQMLMRLGGGTTVTSS

SEQ ID NO: 9 - protein, 1141 aa, Homo sapiens
MDDSGELGGLETMETLTELGDELTLGDIDEMLQFVSNQVGEFPDLFSEQLCSSFPGSGGSGSSSGSSGSSSSSSNGRGSSSGAVDPSVQRSFTQVTLPSFSPSAASPQAPTLQVKVSPTSVPTTPRATPILQPRPQPQPQPQTQLQQQTVMITPTFSTTPQTRIIQQPLIYQNAATSFQVLQPQVQSLVTSSQVQPVTIQQQVQTVQAQRVLTQTANGTLQTLAPATVQTVAAPQVQQVPVLVQPQIIKTDSLVLTTLKTDGSPVMAAVQNPALTALTTPIQTAALQVPTLVGSSGTILTTMPVMMGQEKVPIKQVPGGVKQLEPPKEGERRTTHNIIEKRYRSSINDKIIELKDLVMGTDAKMHKSGVLRKAIDYIKYLQQVNHKLRQENMVLKLANQKNKLLKGIDLGSLVDNEVDLKIEDFNQNVLLMSPPASDSGSQAGFSPYSIDSEPGSPLLDDAKVKDEPDSPPVALGMVDRSRILLCVLTFLCLSFNPLTSLLQWGGAHDSDQHPHSGSGRSVLSFESGSGGWFDWMMPTLLLWLVNGVIVLSVFVKLLVHGEPVIRPHSRSSVTFWRHRKQADLDLARGDFAAAAGNLQTCLAVLGRALPTSRLDLACSLSWNVIRYSLQKLRLVRWLLKKVFQCRRATPATEAGFEDEAKTSARDAALAYHRLHQLHITGKLPAGSACSDVHMALCAVNLAECAEEKIPPSTLVEIHLTAAMGLKTRCGGKLGFLASYFLSRAQSLCGPEHSAVPDSLRWLCHPLGQKFFMERSWSVKSAAKESLYCAQRNPADPIAQVHQAFCKNLLERAIESLVKPQAKKKAGDQEEESCEFSSALEYLKLLHSFVDSVGVMSPPLSRSSVLKSALGPDIICRWWTSAITVAISWLQGDDAAVRSHFTKVERIPKALEVTESPLVKAIFHACRAMHASLPGKADGQQSSFCHCERASGHLWSSLNVSGATSDPALNHVVQLLTCDLLLSLRTALWQKQASASQAVGETYHASGAELAGFQRDLGSLRRLAHSFRPAYRKVFLHEATVRLMAGASPTRTHQLLEHSLRRRTTQSTKHGEVDAWPGQRERATAILLACRHLPLSFLSSPGQRAVLLAEAARTLEKVGDRRSCNDCQQMIVKLGGGTAIAAS

SEQ ID NO: 10 - protein, 350 aa, Homo sapiens
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGIRNVLLAFAGVAGVGAGLAYLIR

SEQ ID NO: 11 - protein, 2174 aa, Homo sapiens
MSASFVPNGASLEDCHCNLFCLADLTGIKWKKYVWQGPTSAPILFPVTEEDPILSSFSRCLKADVLGVWRRDQRPGRRELWIFWWGEDPSFADLIHHDLSEEEDGVWENGLSYECRTLLFKAVHNLLERCLMNRNFVRIGKWFVKPYEKDEKPINKSEHLSCSFTFFLHGDSNVCTSVEINQHQPVYLLSEEHITLAQQSNSPFQVILCPFGLNGTLTGQAFKMSDSATKKLIGEWKQFYPISCCLKEMSEEKQEDMDWEDDSLAAVEVLVAGVRMIYPACFVLVPQSDIPTPSPVGSTHCSSSCLGVHQVPASTRDPAMSSVTLTPPTSPEEVQTVDPQSVQKWVKFSSVSDGFNSDSTSHHGGKIPRKLANHVVDRVWQECNMNRAQNKRKYSASSGGLCEEATAAKVASWDFVEATQRTNCSCLRHKNLKSRNAGQQGQAPSLGQQQQILPKHKTNEKQEKSEKPQKRPLTPFHHRVSVSDDVGMDADSASQRLVISAPDSQVRFSNIRTNDVAKTPQMHGTEMANSPQPPPLSPHPCDVVDEGVTKTPSTPQSQHFYQMPTPDPLVPSKPMEDRIDSLSQSFPPQYQEAVEPTVYVGTAVNLEEDEANIAWKYYKFPKKKDVEFLPPQLPSDKFKDDPVGPFGQESVTSVTELMVQCKKPLKVSDELVQQYQIKNQCLSAIASDAEQEPKIDPYAFVEGDEEFLFPDKKDRQNSEREAGKKHKVEDGTSSVTVLSHEEDAMSLFSPSIKQDAPRPTSHARPPSTSLIYDSDLAVSYTDLDNLFNSDEDELTPGSKKSANGSDDKASCKESKTGNLDPLSCISTADLHKMYPTPPSLEQHIMGFSPMNMNNKEYGSMDTTPGGTVLEGNSSSIGAQFKIEVDEGFCSPKPSEIKDFSYVYKPENCQILVGCSMFAPLKTLPSQYLPPIKLPEECIYRQSWTVGKLELLSSGPSMPFIKEGDGSNMDQEYGTAYTPQTHTSFGMPPSSAPPSNSGAGILPSPSTPRFPTPRTPRTPRTPRGAGGPASAQGSVKYENSDLYSPASTPSTCRPLNSVEPATVPSIPEAHSLYVNLILSESVMNLFKDCNFDSCCICVCNMNIKGADVGVYIPDPTQEAQYRCTCGFSAVMNRKFGNNSGLFLEDELDIIGRNTDCGKEAEKRFEALRATSAEHVNGGLKESEKLSDDLILLLQDQCTNLFSPFGAADQDPFPKSGVISNWVRVEERDCCNDCYLALEHGRQFMDNMSGGKVDEALVKSSCLHPWSKRNDVSMQCSQDILRMLLSLQPVLQDAIQKKRTVRPWGVQGPLTWQQFHKMAGRGSYGTDESPEPLPIPTFLLGYDYDYLVLSPFALPYWERLMLEPYGSQRDIAYVVLCPENEALLNGAKSFFRDLTAIYESCRLGQHRPVSRLLTDGIMRVGSTASKKLSEKLVAEWFSQAADGNNEAFSKLKLYAQVCRYDLGPYLASLPLDSSLLSQPNLVAPTSQSLITPPQMTNTGNANTPSATLASAASSTMTVTSGVAISTSVATANSTLTTASTSSSSSSNLNSGVSSNKLPSFPPFGSMNSNAAGSMSTQANTVQSGQLGGQQTSALQTAGISGESSSLPTQPHPDVSESTMDRDKVGIPTDGDSHAVTYPPAIVVYIIDPFTYENTDESTNSSSVWTLGLLRCFLEMVQTLPPHIKSTVSVQIIPCQYLLQPVKHEDREIYPQHLKSLAFSAFTQCRRPLPTSTNVKTLTGFGPGLAMETALRSPDRPECIRLYAPPFILAPVKDKQTELGETFGEAGQKYNVLFVGYCLSHDQRWILASCTDLYGELLETCIINIDVPNRARRKKSSARKFGLQKLWEWCLGLVQMSSLPWRVVIGRLGRIGHGELKDWSCLLSRRNLQSLSKRLKDMCRMCGISAADSPSILSACLVAMEPQGSFVIMPDSVSTGSVFGRSTTLNMQTSQLNTPQDTSCTHILVFPTSASVQVASATYTTENLDLAFNPNNDGADGMGIFDLLDTGDDLDPDIINILPASPTGSPVHSPGSHYPHGGDAGKGQSTDRLLSTEPHEEVPNILQQPLALGYFVSTAKAGPLPDWFWSACPQAQYQCPLFLKASLHLHVPSVQSDELLHSKHSHPLDSNQTSDVLRFVLEQYNALSWLTCDPATQDRRSCLPIHFVVLNQLYNFIMNML

SEQ ID NO: 12 - protein, 457 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKKYRRDSASVVDQFFTDTEGLPYSINMNVFLPDITHLRTGLYKSQRPCVTHIKTEPVAIFSHQSETTAPPPAPTQALPEFTSIFSSHQTAAPEVNNIFIKQELPTPDLHLSVPTQQGHLYQLLNTPDLDMPSSTNQTAAMDTLNVSMSAAMAGLNTHTSAVPQTAVKQFQGMPPCTYTMPSQFLPQQATYFPPSPPSSEPGSPDRQAEMLQNLTPPPSYAATIASKLAIHNPNLPTTLPVNSQNIQPVRYNRRSNPDLEKRRIHYCDYPGCTKVYTKSSHLKAHLRTHTGEKPYKCTWEGCDWRFARSDELTRHYRKHTGAKPFQCGVCNRSFSRSDHLALHMKRHQN

SEQ ID NO: 13 - protein, 355 aa, Homo sapiens
MALSEPILPSFSTFASPCRERGLQERWPRAEPESGGTDDDLNSVLDFILSMGLDGLGAEAAPEPPPPPPPPAFYYPEPGAPPPYSAPAGGLVSELLRPELDAPLGPALHGRFLLAPPGRLVKAEPPEADGGGGYGCAPGLTRGPRGLKREGAPGPAASCMRGPGGRPPPPPDTPPLSPDGPARLPAPGPRASFPPPFGGPGFGAPGPGLHYAPPAPPAFGLFDDAAAAAAALGLAPPAARGLLTPPASPLELLEAKPKRGRRSWPRKRTATHTCSYAGCGKTYTKSSHLKAHLRTHTGEKPYHCNWDGCGWKFARSDELTRHYRKHTGHRPFQCHLCDRAFSRSDHLALHMKRHM

SEQ ID NO: 14 - protein, 230 aa, Homo sapiens [sequence omitted]

SEQ ID NO: 15 - protein, 401 aa, Homo sapiens
MVLAQSRVSAGVGSPHCSGSGGGGSDSFPWPASHPGNPQCSFSTAFLASPRLSRGTLAYLPPAPWSSLATPSALLGSSCAPPPPPARCPQPRALSPELGTKAGPRRPHRWELPRSPSQGAQGPAPRRRLLETMKGIVAASGSETEDEDSMDIPLDLSSSAGSGKRRRRGNLPKESVQILRDWLYEHRYNAYPSEQEKALLSQQTHLSTLQVCNWFINARRRLLPDMLRKDGKDPNQFTISRRGAKISETSSVESVMGIKNFMPALEETPFHSCTAGPNPTLGRPLSPKPSSPGSVLARPSVICHTTVTALKDVPFSLCQSVGVGQNTDIQQIAAKNFTDTSLMYPEDTCKSGPSTNTQSGLFNTPPPTPPDLNQDFSGFQLLVDVALKRAAEMELQAKLTA

SEQ ID NO: 16 - protein, 480 aa, Homo sapiens
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 17 - protein, 443 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 18 - protein, 480 aa, Homo sapiens
MLNFGASLQQTAEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 19 - protein, 512 aa, Homo sapiens
MHTPDFAGPDDARAVDIMDICESILERKRHDSERSTCSILEQTDMEAVEALVCMSSWGQRSQKGDLLRIRPLTPVSDSGDVTTTVHMDAATPELPKDFHSLSTLCITPPQSPDLVEPSTRTPVSPQVTDSKACTATDVLQSSAVVARALSGGAERGLLGLEPVPSSPCRAKGTSVIRHTGESPAACFPTIQTPDCRLSDSREGEEQLLGHFETLQDTHLTDSLLSTNLVSCQPCLHKSGGLLLTDKGQQAGWPGAVQTCSPKNYENDLPRKTTPLISVSVPAPPVLCQMIPVTGQSSMLPAFLKPPPQLSVGTVRPILAQAAPAPQPVFVGPAVPQGAVMLVLPQGALPPPAPCAANVMAAGNTKLLPLAPAPVFITSSQNCVPQVDFSRRRNYVCSFPGCRKTYFKSSHLKAHLRTHTGEKPFNCSWDGCDKKFARSDELSRHRRTHTGEKKFVCPVCDRRFMRSDHLTKHARRHMTTKKIPGWQAEVGKLNRIASAESPGSPLVSMPASA

SEQ ID NO: 20 - protein, 358 aa, Homo sapiens
MESADFYEAEPRPPMSSHLQSPPHAPSSAAFGFPRGAGPAQPPAPPAAPEPLGGICEHETSIDISAYIDPAAFNDEFLADLFQHSRQQEKAKAAVGPTGGGGGGDFDYPGAPAGPGGAVMPGGAHGPPPGYGCAAAGYLDGRLEPLYERVGAPALRPLVIKQEPREEDEAKQLALAGLFPYQPPPPPPPSHPHPHPPPAHLAAPHLQFQIAHCGQTTMHLQPGHPTPPPTPVPSPHPAPALGAAGLPGPGSALKGLGAAHPDLRASGGSGAGKAKKSVDKNSNEYRVRRERNNIAVRKSRDKAKQRNVETQQKVLELTSDNDRLRKRVEQLSRELDTLRGIFRQLPESSLVKAMGNCA